SEQUENCE LISTING (3 sequences)

SEQ ID NO: 1 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 2 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 3 - protein, 138 aa, Artificial Sequence
  interferon gamma
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

(19) United States
(12) Patent Application Publication
(10) Pub. No.: US 2014/0039158 A1
(43) Pub. Date: Feb. 6, 2014

(54) POLYMERIC CONJUGATES AND METHODS OF PREPARING THE SAME

(71) Applicant: Belrose Pharma, Inc., Princeton, NJ (US)

(72) Inventor: Hong Zhao, Edison, NJ (US)

(73) Assignee: Belrose Pharma, Inc., Princeton, NJ (US)

(21) Appl. No.: 13/951,238

(22) Filed: Jul. 25, 2013

Related U.S. Application Data
(60) Provisional application No. 61/675,698, filed on Jul. 25, 2012.

(51) Int. Cl. C07K 14/57

(52) U.S. Cl. CPC C07K 14/57
USPC 530/351; 530/397; 530/391.9

(57) ABSTRACT

Methods of preparing polymer target conjugates which are substantially free of polymer attachment on the N-terminal of the targets are provided. Also provided are compositions comprising a plurality of polymer-polypeptide conjugates, said polymer-polypeptide conjugate comprising a polypeptide covalently attached to at least one polymer through an epsilon amino group of a Lysine or a Histidine found on the polypeptide and said conjugates containing less than 5% of the polymer-polypeptide conjugates having a polymer attached to the N-terminal of the polypeptide; and polymer target conjugates comprising a target moiety selected from the group consisting of polypeptides, proteins and the like having at least one polymer attached thereto at a site which is not the N-terminal of the target.

### CROSS-REFERENCE TO RELATED APPLICATION

This application claims the benefit of priority from U.S. Provisional Patent Application Ser. No. 61/675,698 filed Jul. 25, 2012, the contents of which are incorporated herein by reference.

### BACKGROUND OF THE INVENTION

Interferon-Gamma is a biologically active dimer is formed by anti-parallel inter-locking of the two monomers as shown in red and blue. Cellular responses to IFN-gamma are activated through its interaction with a heterodimeric receptor consisting of Interferon gamma receptor 1 and 2 (see below in green and black, respectively).

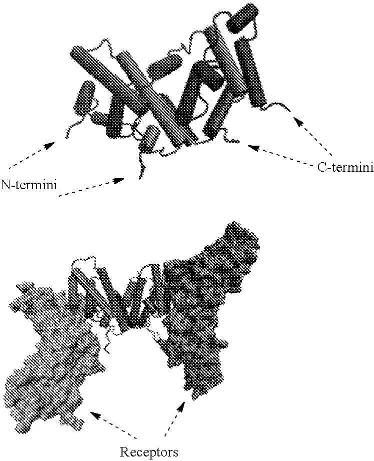

Exist as a dimer with 6 alpha-helices and no beta-sheet, noncovalently bound in an antiparalled orientation.)
Hydrophilic C-terminus binds to a region of the receptor distinct from the N-terminus likely through the polycationic region (Griggs, al. et. J Immunology 1992, Vol. 149. No. 2. Page 517-520).

Interferons are secreted by infected cells to warn their neighbors, and once stimulated, cells of the immune system secrete interferons as part of their viral surveillance. Interferons are small proteins that bind to receptors on the cell surface. This signal is transmitted into the cells and leads to production of hundreds of proteins involved in viral defense. Several types of interferons are made by our cells. Interferon-alpha and interferon-beta, shown here from PDB entries 1itf and 1au1, are the most common types, and are made by most types of cells, especially cells of the immune system. They send a basic signal to stop growing and focus on defense. Interferon-gamma, shown here from PDB entry 1rfb, is secreted primarily by T-cells, and sends signals that tune the response of the immune system.

ACTIMMUNE® (Interferon gamma-1b), a biologic response modifier, is a single-chain polypeptide containing 140 amino acids. Production of ACTIMMUNE (interferon gamma 1b) is achieved by fermentation of a genetically engineered Escherichia coli bacterium containing the DNA which encodes for the human protein. Purification of the product is achieved by conventional column chromatography. ACTIMMUNE (interferon gamma 1b) is a highly purified sterile solution consisting of non-covalent dimers of two identical 16,465 dalton monomers; with a specific activity of 20 million International Units (IU)/mg (2×10⁶ IU per 0.5 mL) which is equivalent to 30 million units/mg.

In spite of previous efforts, there is still an unmet need for an improved method of preparing a polymer target conjugates which are substantially free of polymer attachment on the N-terminal of said target. For example, it was reported that both N- and C-termini were involved in receptor binding. Because interferon gamma exists as a dimer, PEGylation at N- or C-terminus may or may not affect the receptor binding. Also, in order to provide the desired improvement the present invention also addresses the need for a polymer target conjugate comprising a target moiety selected from polypeptides, proteins and the like having at least one polymer attached thereto at a site which is not the N-terminal of the target.

### SUMMARY OF THE INVENTION

In accordance with preferred aspects of the invention, there are provided methods of preparing polymer target conjugates which are substantially free of polymer attachment on the N-terminal of the targets. For purpose of the present invention “target” shall be understood to include proteins, peptides, and the like. The methods include

(a) reacting a target with a capping reagent under conditions to selectively cap the N-terminal of the target;

(b) reacting the N-terminal-capped target with an activated polymer under conditions to allow polymer attachment to at least one site on the N-terminal-capped target to form a polymer-target conjugate which is substantially free of polymer attachment on the N-terminal of said target.

In many instances the invention is described with respect to interferon-gamma as the illustrative species. It will be appreciated that the methods described herein are equally useful for the preparation of polymer conjugates of proteins, other peptides, antibodies, immunotoxins, antibody fragments, blood factors, etc, all of which have an N-terminal in their amino acid sequence. The methods allow conjugates of such targets to be made with essentially no polymer attachment to the N-terminals thereof.

### BRIEF DESCRIPTION OF THE DRAWINGS

FIG. 1 provides the preparation of N-terminal protected IFN-gamma compounds 1-7;

FIG. 2 provides the preparation of N-terminal protected IFN-gamma compound 8;

FIG. 3 provides the preparation of N-terminal protected IFN-gamma compounds 1a-7a;

FIG. 4 provides the preparation of N-terminal protected IFN-gamma compound 8a;

FIG. 5 provides the preparation of Lys or His PEGylated IFN-gamma compounds 11-17;

FIG. 6 provides the preparation of Lys or His rPEGylated IFN-gamma compound 18;

FIG. 7 provides the preparation of Lys or His scPEGylated IFN-gamma compounds 21-27;

FIG. 8 provides the preparation of Lys or His scPEGylated IFN-gamma compounds 21a-27a;

FIG. 9 provides the preparation of Lys or His tPEGylated IFN-gamma compounds 31-37;

FIG. 10 provides the preparation of Lys or His tPEGylated IFN-gamma compounds 31a-37a;

FIG. 11 provides the preparation of Lys or His B-PEGylated IFN-gamma compounds 41-47;

FIG. 12 provides the preparation of Lys or His B-PEGylated IFN-gamma compounds 41a-47a; and

FIG. 13 provides the structures of B-PEG of FIGS. 11 and 12.

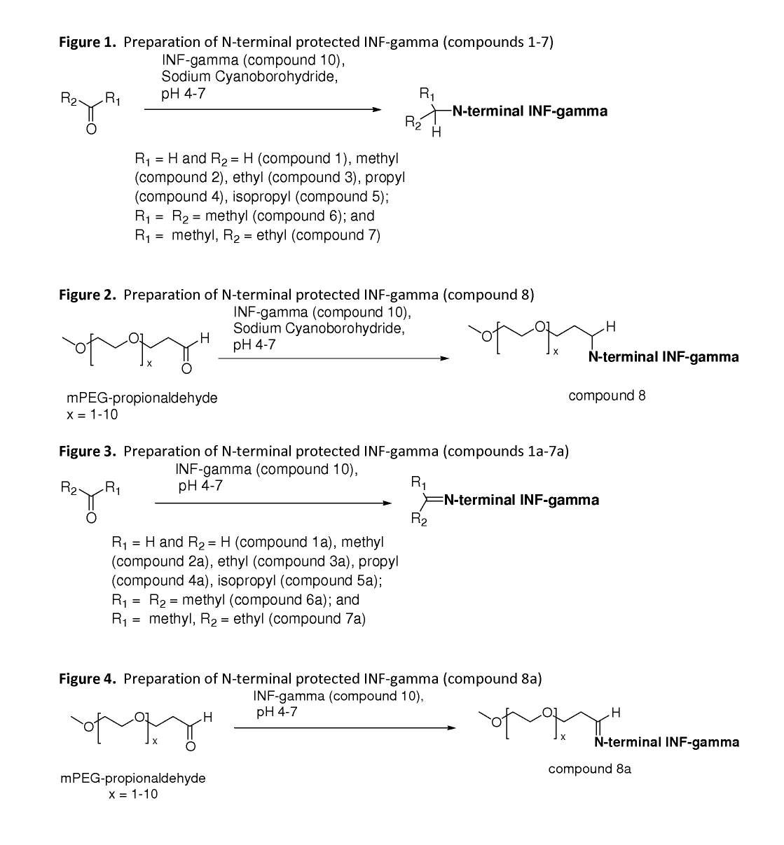

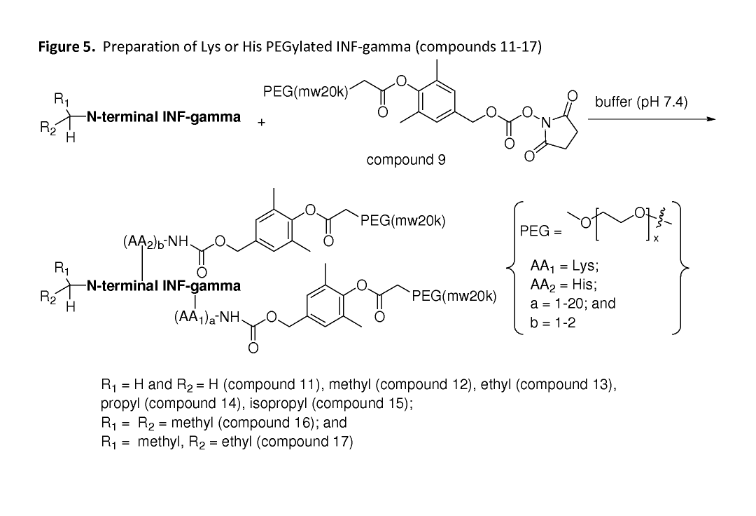

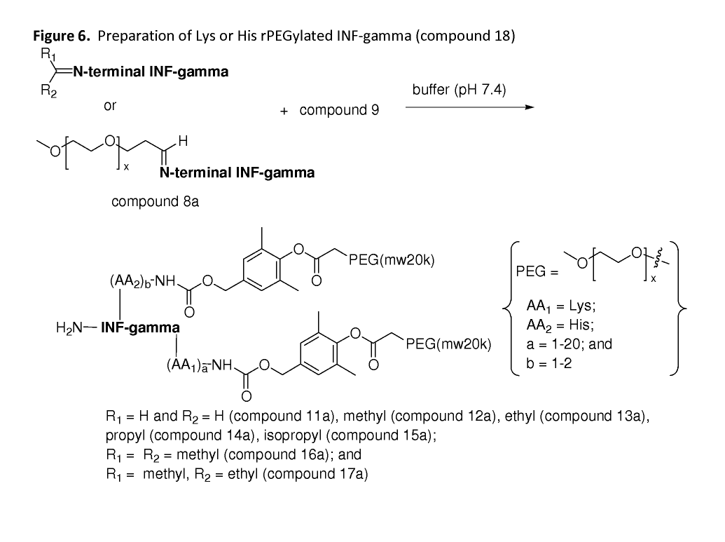

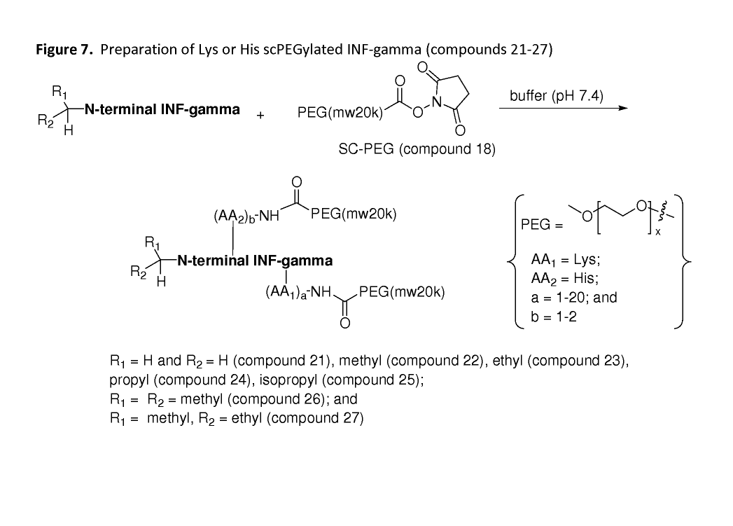

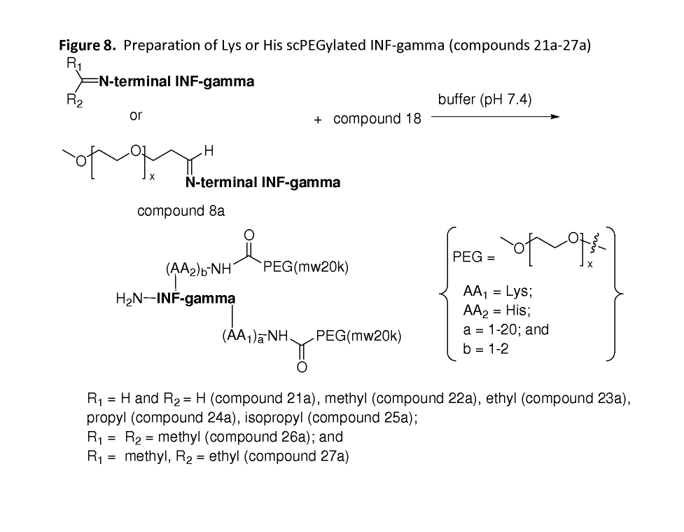

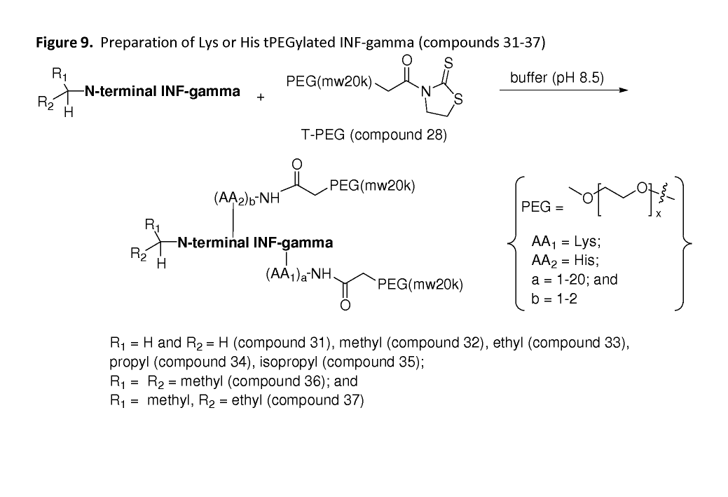

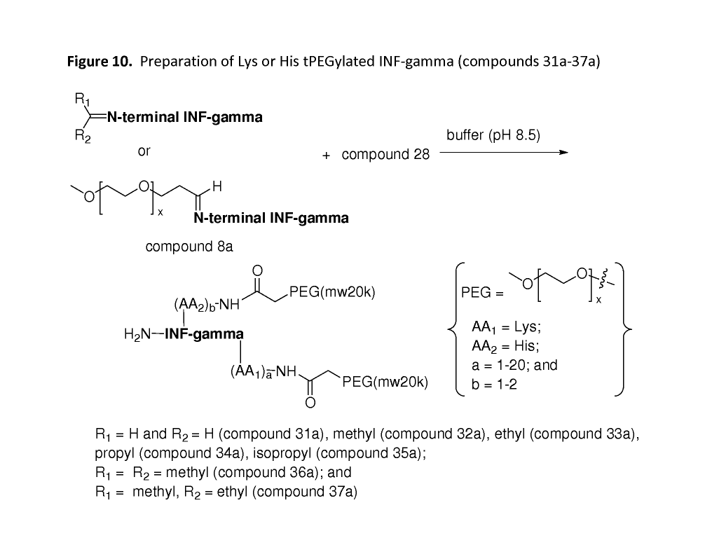

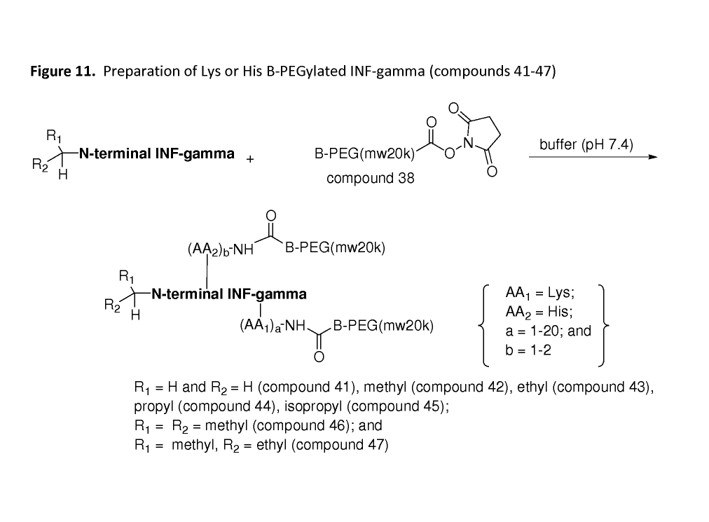

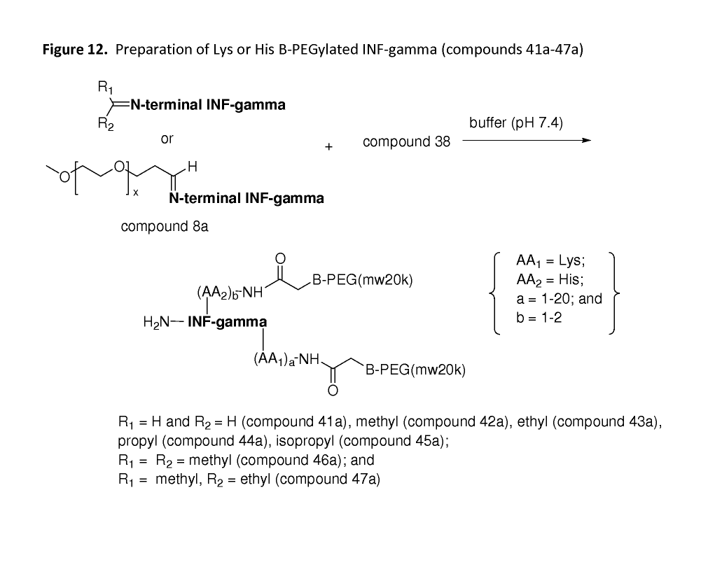

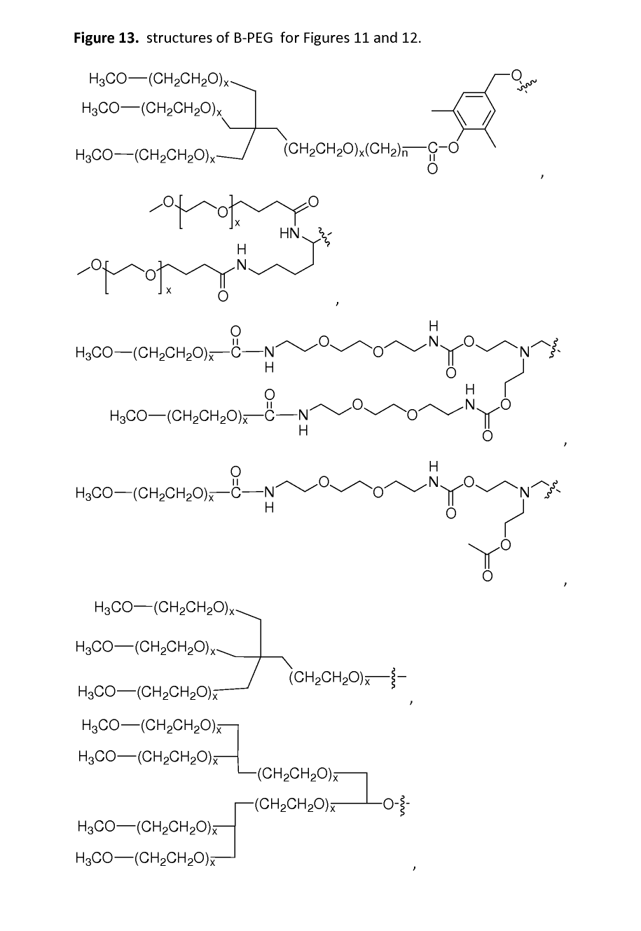

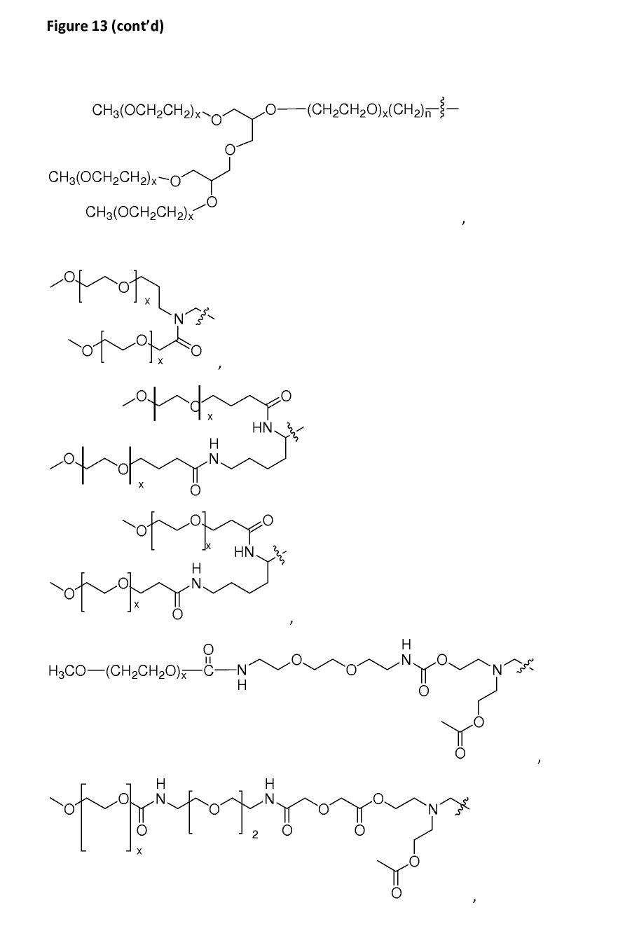

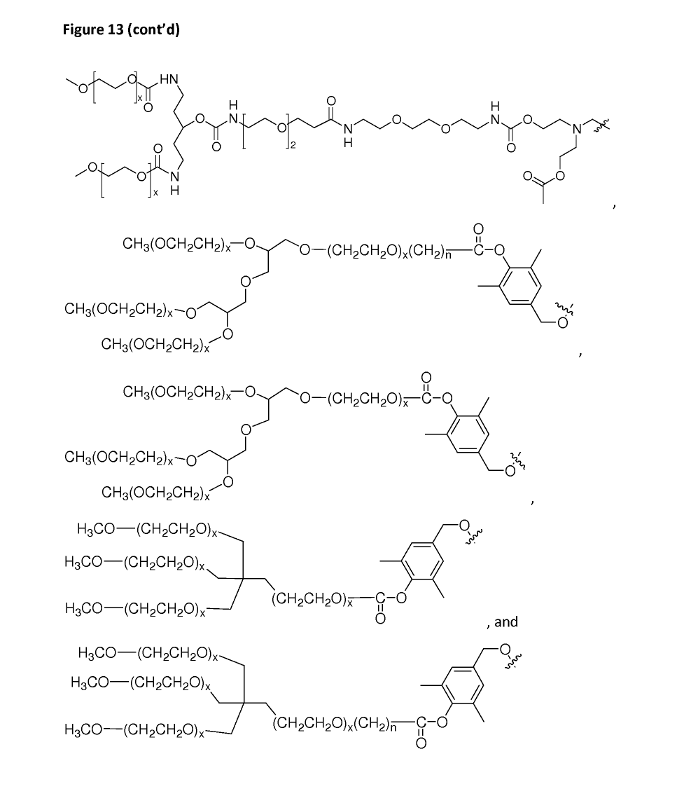

### DETAILED DESCRIPTION OF THE INVENTION

In accordance with the invention, there are provided the following embodiments:

Embodiment 1: a method of preparing a polymer target conjugate which is substantially free of polymer attachment on the N-terminal of said target, said target being selected from the group consisting of proteins, peptides, and the like, comprising:

(a) reacting a target with a capping reagent under conditions to selectively cap the N-terminal of the target;

(b) reacting the N-terminal-capped target with an activated polymer under conditions to allow polymer attachment to at least one site on the N-terminal-capped target to form a polymer-target conjugate which is substantially free of polymer attachment on the N-terminal of said target.

Embodiment 2: the method of embodiment 1 further comprising isolating the polymer-target conjugate which is substantially free of polymer attachment on the N-terminal of said target resulting from step (b).

Embodiment 3: the method of embodiment 1 further comprising deprotecting the N-terminal of the polymer-target conjugate.

Embodiment 4: the method of embodiment 1, wherein said conditions to selectively cap the N-terminal of the target include reacting the target with the capping reagent at pH of from about 4.0 to about 7.0.

Embodiment 5: the method of embodiment 4, wherein said capping reagent is of the formula

R₁—C(═O)—R₂

wherein R₁ and R₂ are independently selected from the group consisting of H, C₁₋₆ alkyl, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, and

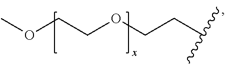

wherein x is an integer selected from the group consisting of 1, 2, 3, 4, 5, 6, 7, 8, 9, and 10.

Embodiment 6: the method of embodiment 5, wherein R₁ is C₁₋₆ alkyl and R₂ is H.

Embodiment 7: the method of embodiment 5, wherein R₁ is CH₃ and R₂ is H.

Embodiment 8: the method of embodiment 5, wherein R₁ is

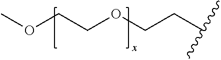

### and R₂ is H.

Embodiment 9: the method of embodiment 4, wherein said conditions further comprise reducing the bond attaching the capping group to the N-terminal of the target with a reducing agent.

Embodiment 10: the method of embodiment 9, wherein the reducing agent is selected from the group consisting of sodium cyanoborohydride (NaBH₃CN), sodium triacetoxyborohydride (NaBH(OC(═O)OCH₃)₃), sodium hydride, decaborane (B₁₀H₁₄), InCl₃-Et₃SiH complex, Nickel nanoparticles, Et₃SiH-iridium complex, and Ti(iOPr)₄.

Embodiment 11: the method of embodiment 10, wherein the reducing agent is sodium cyanoborohydride.

Embodiment 12: the method of embodiment 1, wherein the target is a protein.

Embodiment 13: the method of embodiment 1, wherein the target is a polypeptide.

Embodiment 14: the method of embodiment 13, wherein the target is an interferon.

Embodiment 15: the method of embodiment 14, wherein said interferon is interferon-gamma (IFN-gamma).

Embodiment 16: the method of embodiment 15, wherein the interferon-gamma (IFN-gamma) has SEQ ID NO: 1.

Embodiment 17: the method of embodiment 15, wherein the interferon-gamma (IFN-gamma) has SEQ ID NO: 2 or SEQ ID NO: 3.

Embodiment 18: the method of embodiment 13, wherein the polypeptide is erythropoietin (EPO), immunotoxin antibody or a fragment thereof, blood factors.

Embodiment 19: the method of embodiment 1, wherein the activated polymer is an activated polyethylene glycol (PEG).

Embodiment 20: the method of embodiment 19, wherein the activated PEG includes a leaving group selected from the group consisting of N-hydroxybenzotriazolyl, halogen, N-hydroxy-phthalimidyl, p-nitrophenoxy, imidazolyl, N-hydroxysuccinimidyl; thiazolidinyl thione, O-acyl ureas or

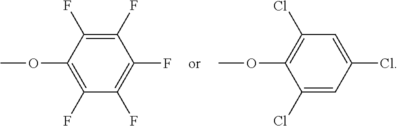

Embodiment 21: the method of embodiment 20, wherein the activated PEG is selected from the group consisting of:

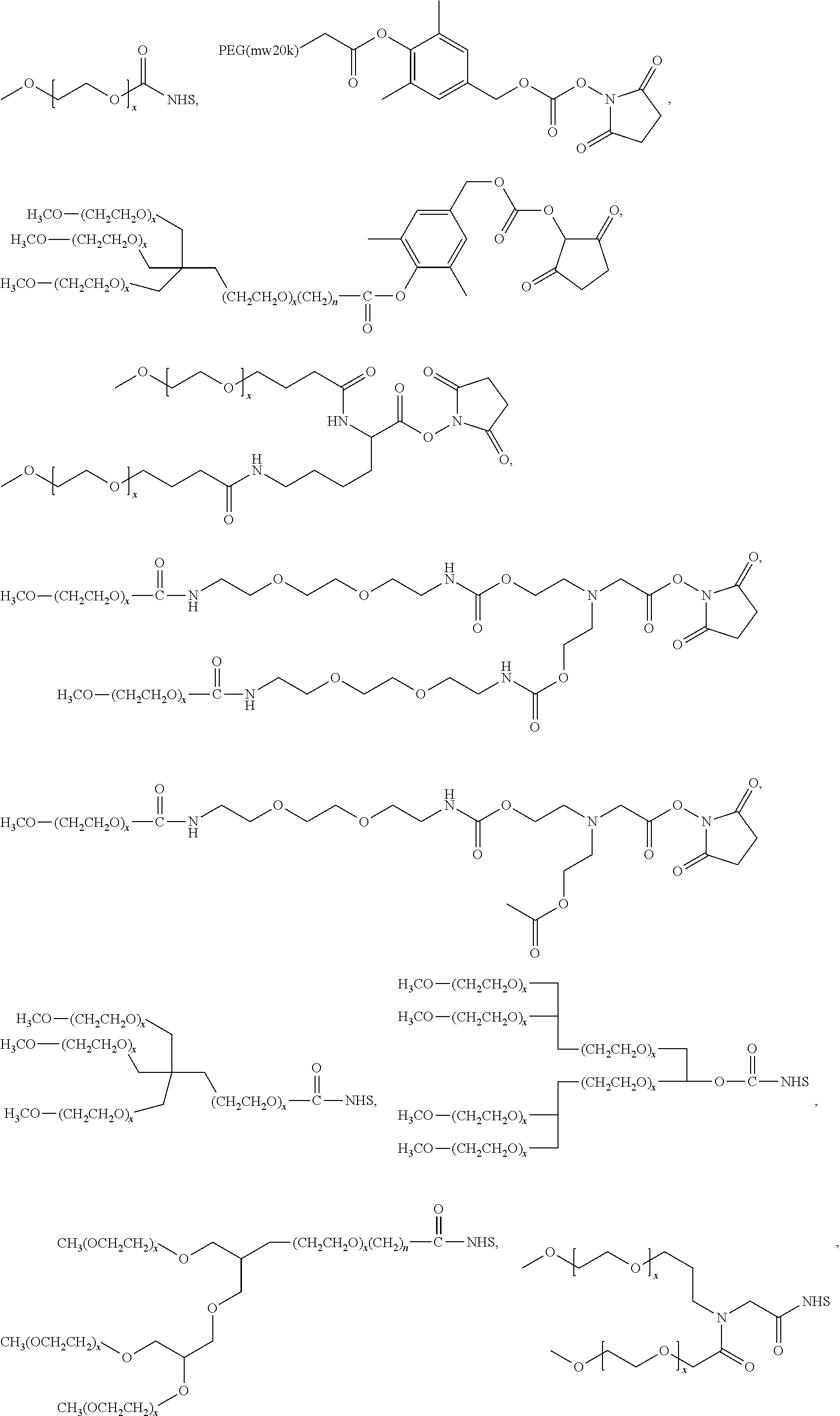

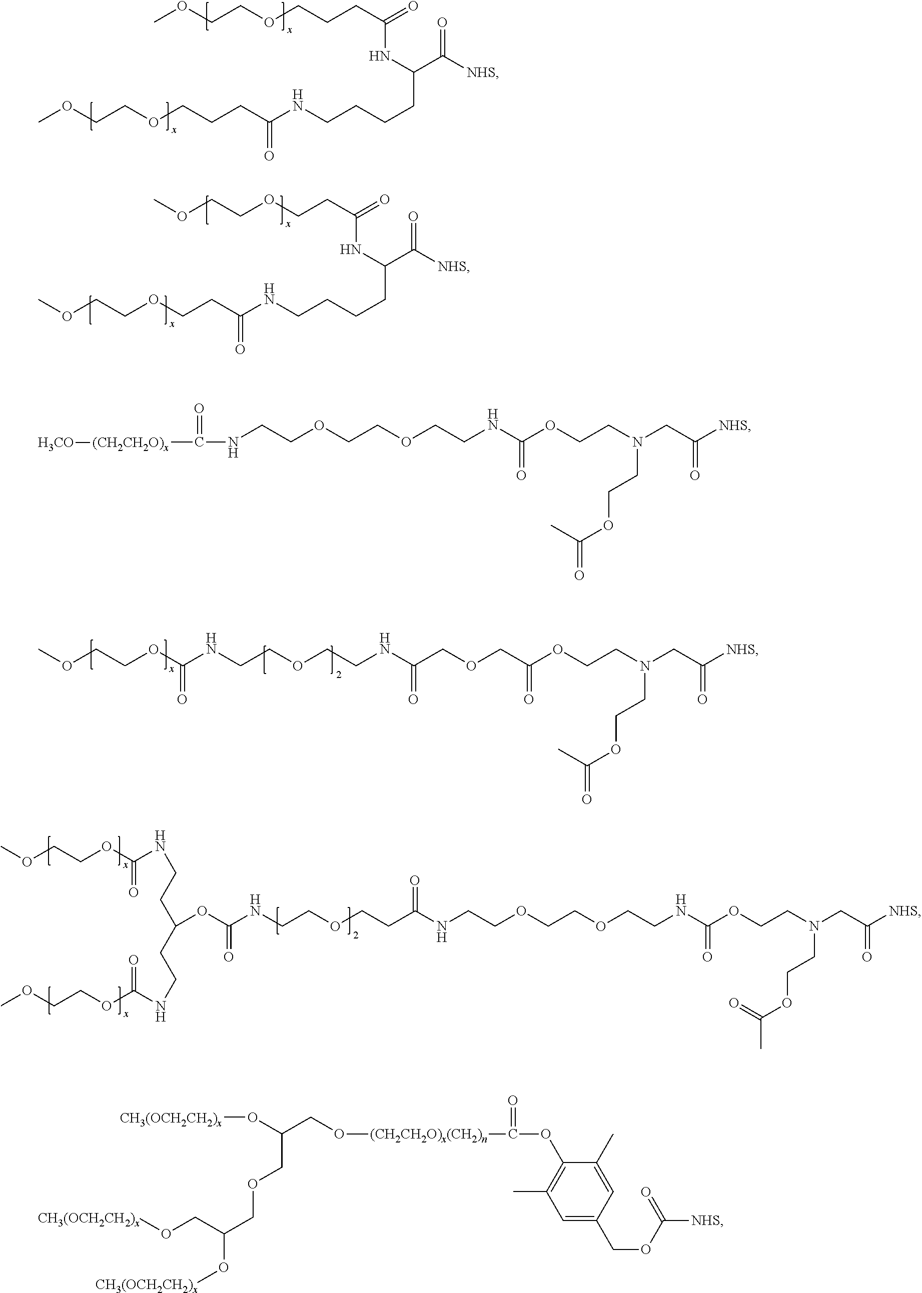

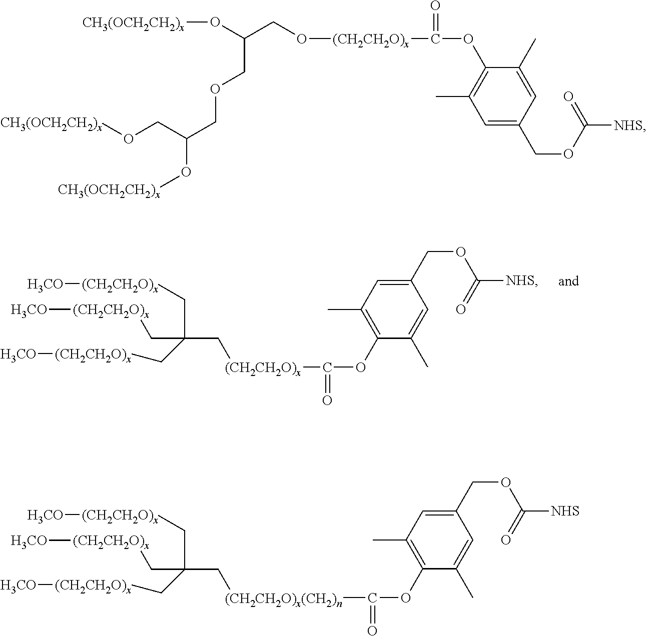

wherein, NHS is

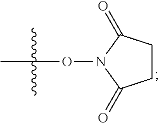 [SMILES: *ON1C(=O)CCC1=O]

(x) is a degree of polymerization positive integer of from about 10 to about 2,300 so that the polymeric portion of the compound has the total number average molecular weight of from about 2,000 to about 100,000 daltons; and

(n) is an integer selected from the group consisting of 1, 2, 3, 4, 5, 6, and 7.

Embodiment 22: the method of embodiment 20, wherein the activated PEG is selected from the group consisting of:

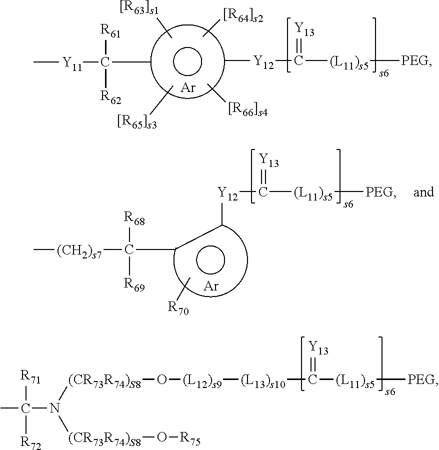

wherein

PEG is a polyethylene glycol;

Y₁₁ is O, or S;

Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;

Y₁₃ is O, S, or NR₆₇;

L₁₁₋₁₃ are independently bifunctional linking moiety selected from the group consisting of
—[C(═O)]ₛ₁₁CR₇₆R₇₇OCR₇₆R₇₇[C(═O)]ₛ₁₂—[Y₁₅]ₛ₁₃—;
—[C(═O)]ₛ₁₁CR₇₆R₇₇NR₇₈CR₇₆R₇₇[C(═O)]ₛ₁₂—[Y₁₅]ₛ₁₃—;
—[C(═O)]ₛ₁₁CR₇₆R₇₇SCR₇₆R₇₇[C(═O)]ₛ₁₂—[Y₁₅]ₛ₁₃—; and
—[C(═O)]ₛ₁₁ (CR₇₆R₇₇)ₛ₁₁[C(═O)]ₛ₁₂—[Y₁₅]ₛ₁₃—;
or C(═Y₁₃)-L11- together form an amino acid;

L₁₄ is a bifunctional linking moiety, and the same as defined as L₁ and L₂;

R₆₁, R₆₂, R₆₇, R₇₁, R₇₂, R₇₃ and R₇₄ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;

R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;

R₇₅ is H, —C(═O)—R₇₉, wherein R₇₉, in each occurrence, is the same or different alkyl,

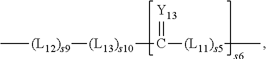

or
a targeting group;

R₇₆, R₇₇ and R₇₈ are independently selected from the group consisting of from H, C₁₋₆ alkyl, C₂₋₆ alkenyl, C₂₋₆ alkynyl, C₁₋₆ heteroalkyl and aryl;

Ar is a moiety which when included in the formula forms an aromatic or heteroaromatic hydrocarbon;

(s1), (s2), (s3), and (s4) are independently zero or one;

(s5) is a positive integer of from about 1 to about 6;

(s6) is zero or a positive integer;

(s7) is zero, one or two;

(s8) is 1, 2 or 3;

(s9) is zero or one;

(s10) is zero or a positive integer; and

(s11), (s12), and (s13) are independently zero or one.

Embodiment 23: the method of embodiment 19, wherein the activated PEG is mPEG.

Embodiment 24: the method of embodiment 19, wherein the activated PEG is selected from the group consisting of:

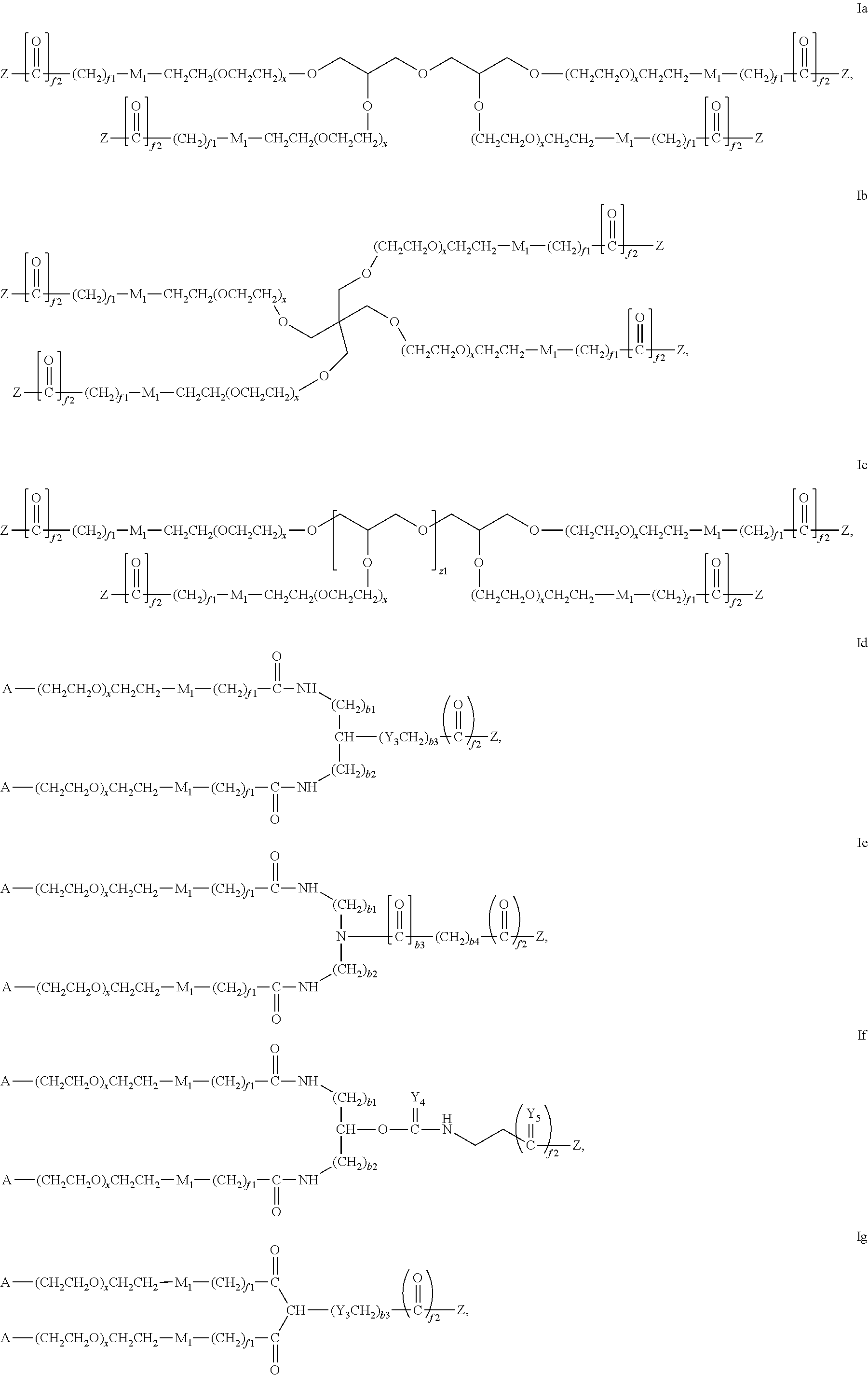

(Ih) Z—[C(═O)]_{f2}—(CH₂)_{f1}-M₁-CH₂CH₂—O—(CH₂CH₂O)ₓ—CH₂CH₂-M₁-(CH₂)_{f1}—[C(═O)]_{f2}—Z, and
(Ii) A-(CH₂CH₂O)ₓ—CH₂CH₂-M₁-(CH₂)_{f1}—[C(═O)]_{f2}—Z,

wherein

A is hydroxyl, NH₂, CO₂H, or C₁₋₆ alkoxy;

M₁ is O, S, or NH;

Y₃ is O, NR₅₁, S, SO or SO₂;

Y₄ and Y₅ are independently O, S or NR₅₁;

R₅₁, in each occurrence, is independently hydrogen, C₁₋₈ alkyl, C₁₋₈ branched alkyl, C₁₋₈ substituted alkyl, aryl, or aralkyl;

Z, in each occurrence, is independently OH, a leaving group, an activating group;

(b1) and (b2) are independently zero or positive integers;

(b3) is zero or 1;

(b4) is a positive integer;

(f1) is zero or a positive integer of from about 1 to about 10;

(f2) is zero or 1;

(z1) is zero or a positive integer of from 1 to about 27;

(x) is a degree of polymerization positive integer of from about 10 to about 2,300 so that the polymeric portion of the compound has the total number average molecular weight of from about 2,000 to about 100,000 daltons, provided that one or more Z is a leaving group.

Embodiment 25: the method of embodiment 1, wherein the conditions to allow polymer attachment include reacting the activated polymer with the N-terminal capped target in the presence of an aqueous buffer having a pH of from about 6.0 to about 10.0.

Embodiment 26: the method of embodiment 26, wherein the pH is from about 7.0 to about 10.0.

Embodiment 27: the method of embodiment 1, wherein the polymer is attached to an epsilon amino group of a lysine.

Embodiment 28: the method of embodiment 1, wherein the polymer is attached to a histidine.

Embodiment 29: the method of embodiment 1, wherein the molecular weight of the activated polymer is from about 2,000 to about 100,000 daltons.

Embodiment 30: the polymer-target conjugate prepared by the method of any of the embodiments 1-29.

Embodiment 31: a polymer target conjugate comprising a target moiety selected from the group consisting of polypeptides, proteins and the like having at least one polymer attached thereto at a site which is not the N-terminal of the target.

Embodiment 32: the polymer target conjugate of embodiment 31, wherein the target is an interferon.

Embodiment 33: the polymer target conjugate of embodiment 32, wherein the interferon is an interferon-gamma (IFN-gamma).

Embodiment 34: the polymer target conjugate of embodiment 32, wherein the polymer is attached to at least one Epsilon amino group of a Lysine or a Histidine of the interferon.

Embodiment 35: the polymer target conjugate of embodiment 31, wherein the target moiety further includes a capping group covalently attached to the N-terminal of the target moiety.

Embodiment 36: a composition comprising a plurality of polymer-polypeptide conjugates, said polymer-polypeptide conjugate comprising a polypeptide covalently attached to at least one polymer through an epsilon amino group of a Lysine or a Histidine found on the polypeptide and said conjugates containing less than 5% of the polymer-polypeptide conjugates having a polymer attached to the N-terminal of the polypeptide.

Embodiment 37: the composition of embodiment 36, wherein said conjugates contain less than 1% of the polymer-polypeptide conjugates having a polymer attached to the N-terminal of the polypeptide.

Embodiment 38: the polymer target conjugate of embodiment 1 selected from the group consisting of:

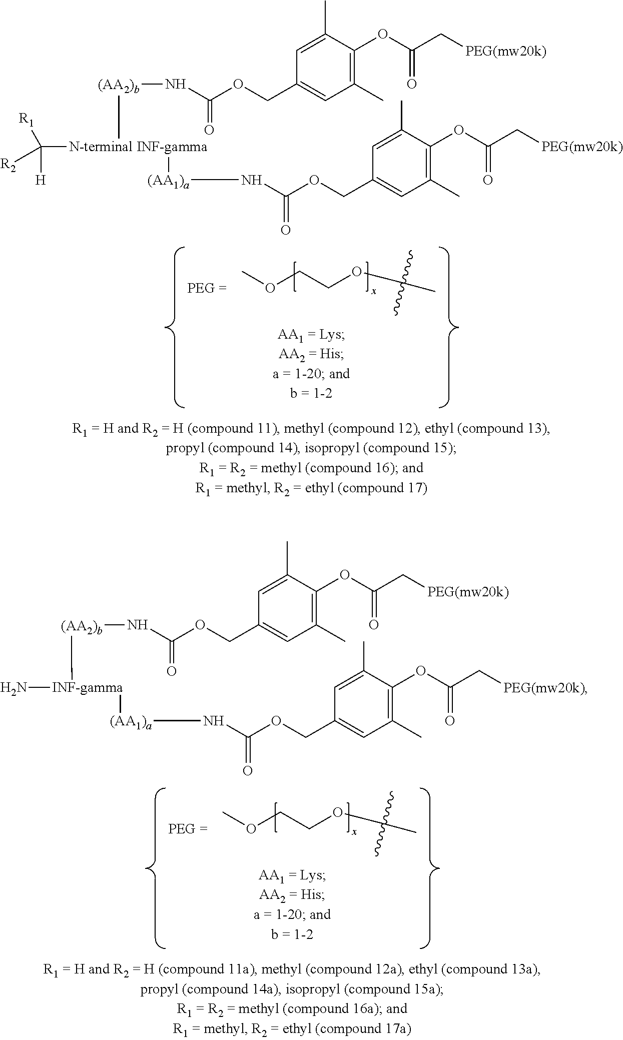

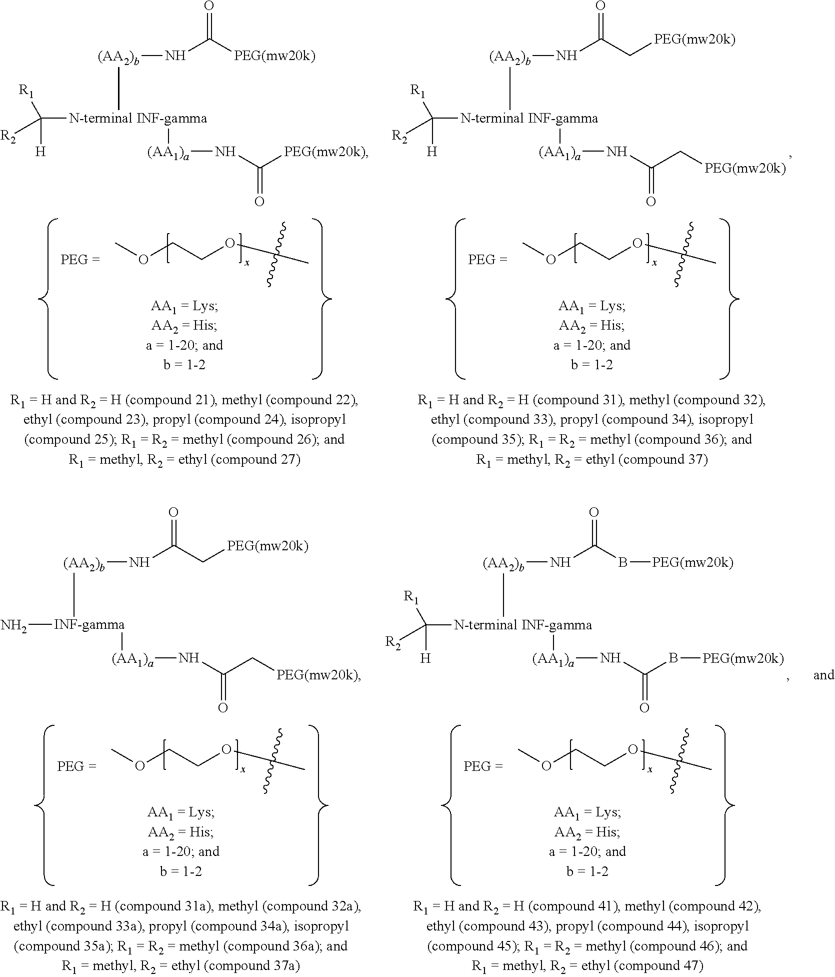

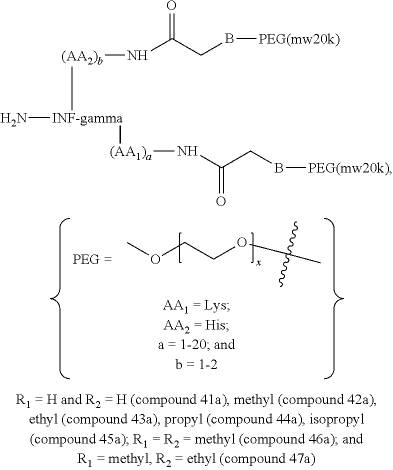

wherein,
(x) is a degree of polymerization positive integer of from about 10 to about 2,300 so that the polymeric portion of the compound has the total number average molecular weight of from about 2,000 to about 100,000 daltons;
B-PEG is selected from the group consisting of:

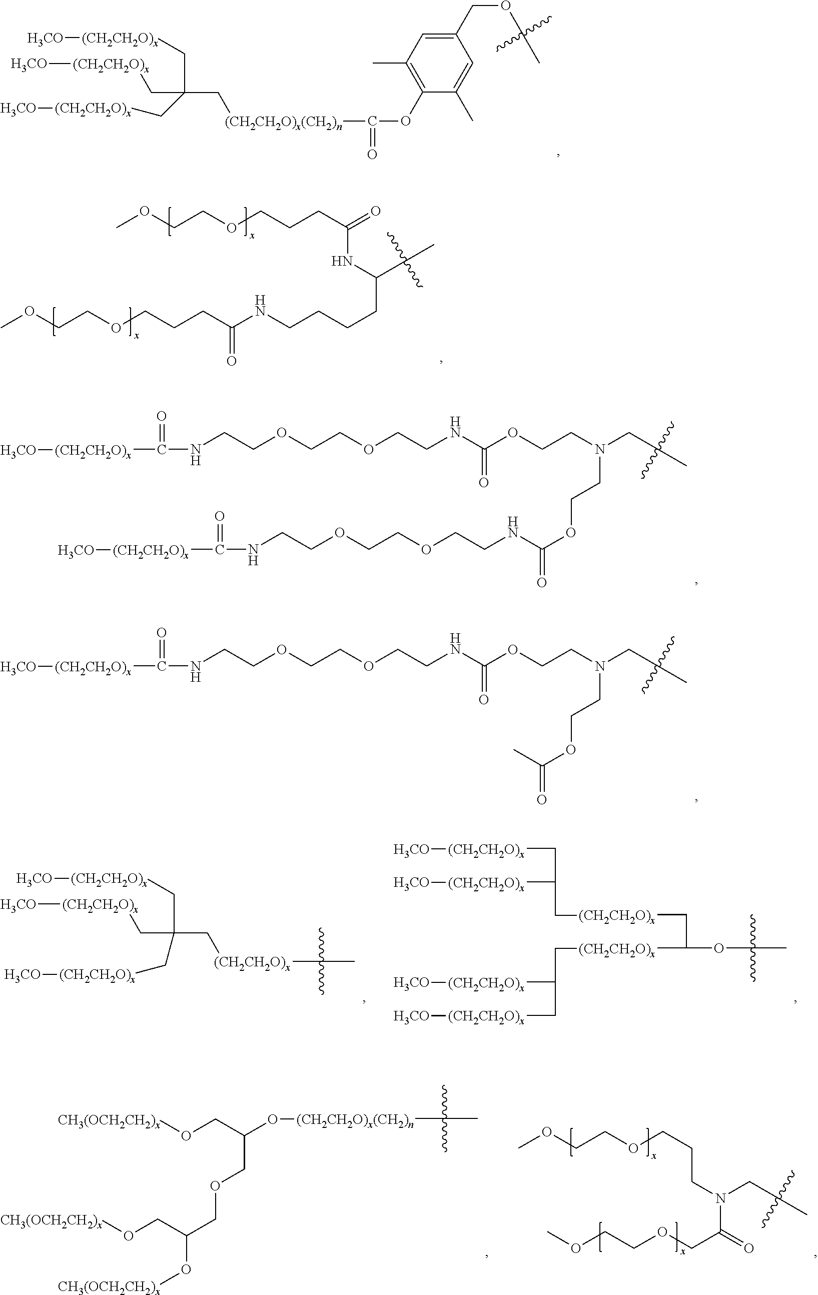

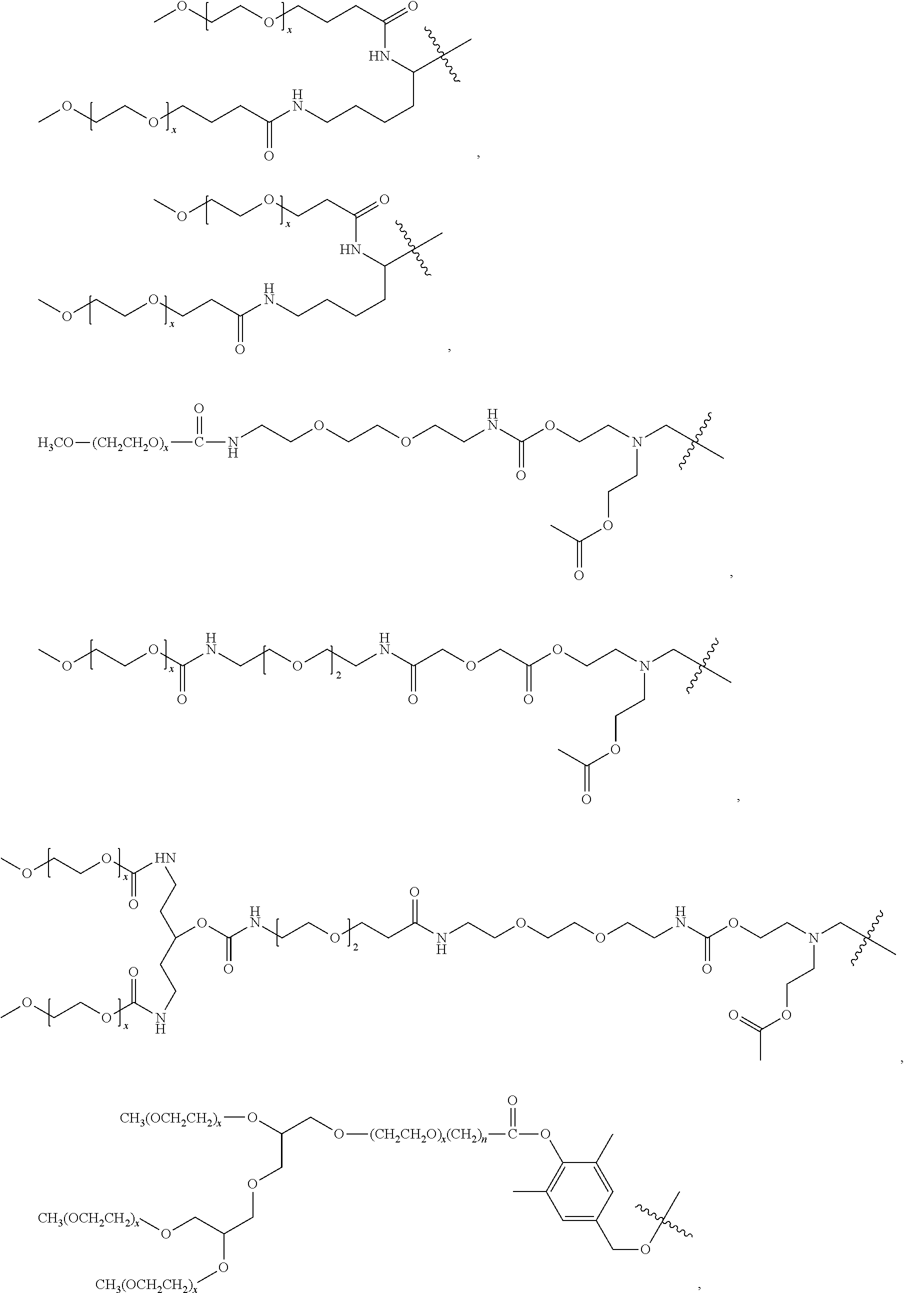

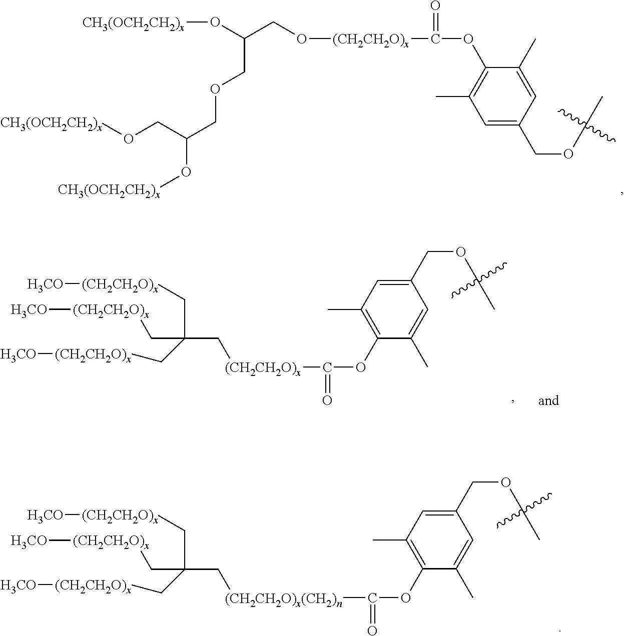

### Targets

As mentioned above, targets can be proteins, polypeptides, other peptides, antibodies, immunotoxins, antibody fragments, blood factors, etc, all of which have an N-terminal in their amino acid sequence.

In one preferred aspect of the invention the target is interferon-gamma.

The method may also include isolating the polymer-target conjugates which are substantially free of polymer attachment on the N-terminal of the target resulting from conjugating step. If desired, the protected N-terminal on the polymer target conjugate can be deprotected as described below.

Some preferred interferon gammas are of SEQ ID NO: 1, SEQ ID NO: 2 or SEQ ID NO: 3.

In another aspect of the invention the polypeptide is erythropoietin (EPO), immunotoxin antibody or a fragment thereof.

Interferon gamma exists as a dimer, PEGylation at N- or C-terminus may or may not affect the receptor binding. Because most lysine residues are away from N- and C-termini, we can protect N-terminus with releasable or permanent groups and PEGylation of epsilon amine groups of lysines provides conjugates with high levels of retainedactivity.

Sequence Analysis of Interferon-Gamma:

(SEQ ID NO: 1)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAEL SPAAKTGKRKRSQMLFRG

Number of amino acids: 138

Molecular weight: 16177.4

Theoretical pl: 9.52.

Ext. coefficients: 0.708 mL/mg.cm
No Cysteine
20 Lysines
There are five very basic propeptides at C-terminus which are RRASQ.
Two glycosylation sites: 48 and 120.

| | Interferon gamma | |
|---|---|---|
| | chain | Actimmune |
| Amino Acid# | 138 (chain) | 146 |
| MW | 16177.4 | 17145.6 |
| pl | 9.52 | 9.54 |
| Cysteine | 0 | 2 |
| Lysine | 20 | 20 |
| Ext. Coefficients: | 0.708 | 0.763 |
| mL/mg.com | | |
| Glycosylation sites | 48 and 120 | No (E. Coli.) |

Notes
There are five basic propeptides at C-terminus which are RRASQ and three a.a. from signal peptide

### Capping Reagents

The capping reagents useful for carrying out the methods described herein can be of the formula

R₁—C(═O)—R₂

wherein R₁ and R₂ are independently selected from the group consisting of H, C₁₋₆ alkyl, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, and

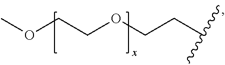

wherein x is an integer selected from among 1, 2, 3, 4, 5, 6, 7, 8, 9, and 10. Preferably R₁ is C₁₋₆ alkyl and R₂ is H. Alternatively, R₁ is CH₃ and R₂ is H. In yet a further aspect of the invention R₁ is

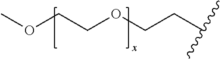

### and R₂ is H.

### Polymers

The polymer in the present invention is, preferably, a substantially non-antigenic polymer, such as polyalkylene oxide. According to the present invention, polymers contemplated within the conjugates described herein are preferably water soluble and substantially non-antigenic, and include, for example, polyalkylene oxides (PAO's). The conjugates described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

PEG is generally represented by the structure:

—(CH₂CH₂O)ₓ—

where (x) is a positive integer of from about 10 to about 2300 so that the polymeric portion of the conjugates described herein has a number average molecular weight of from about 2,000 to about 100,000 daltons.

The polyalkylene oxide has a total number average molecular weight of from about 2,000 to about 100,000 daltons, preferably from about 5,000 to about 60,000 daltons. The molecular weight of the polyalkylene oxide can be more preferably from about 5,000 to about 25,000 or from about 20,000 to about 45,000 daltons. In some particularly preferred embodiments, the conjugates described herein include the polyalkylene oxide having a total number average molecular weight of from about 30,000 to about 45,000 daltons. In one particular embodiment, a polymeric portion has a total number average molecular weight of about 40,000 daltons.

Alternatively, the polyethylene glycol can be further functionalized as represented by the structure:

—[C(═O)]_{f2}—(CH₂)_{f1}-M₁-CH₂CH₂(OCH₂CH₂)ₙ—O-A

wherein

M₁ is O, S, or NH;

(f1) is zero or a positive integer of from about 1 to about 10, preferably, 0, 1, 2, or 3, more preferably, zero or 1;

(f2) is zero or one;

(n) is a positive integer of from about 10 to about 2,300; and

A is hydroxyl, NH₂, CO₂H, or C₁₋₆ alkoxy.

In one embodiment, A is methoxy.

In certain embodiments, all four of the PEG arms can be converted to suitable activating groups, for facilitating attachment to other molecules (e.g., bifunctional linkers). Such conjugates prior to conversion include:

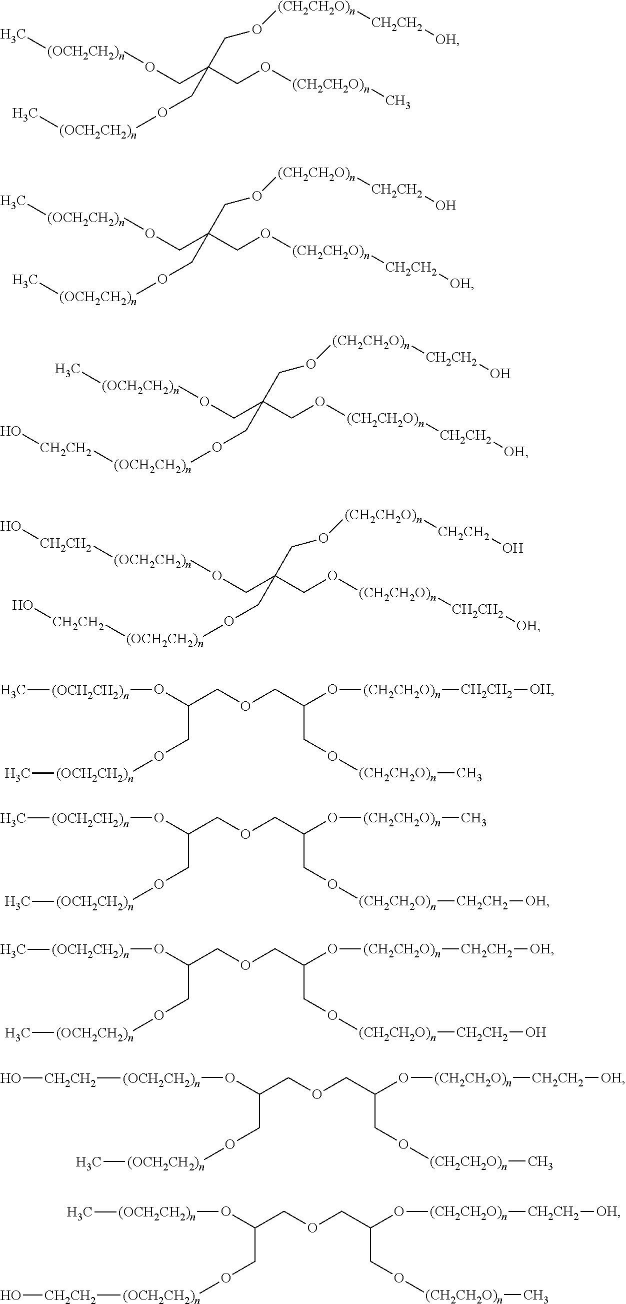

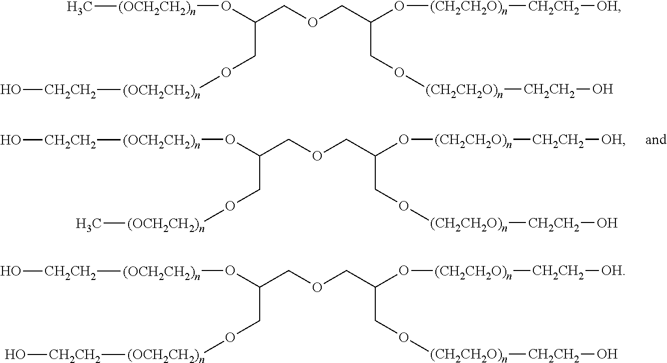

PEG may be conjugated to the target, such as IFN-gamma as described herein, directly or via a linker moiety. The polymers are converted into a suitably activated polymer for conjugation to the target, such as IFN-gamma as described herein, using the activation techniques described in U.S. Pat. Nos. 5,122,614 and 5,808,096 and other techniques known in the art without undue experimentation.

Examples of activating groups for polymers useful for the preparation of a polymer target conjugates in the present invention include a list, but not limited to, aldehyde, carbonyl imidazole, chloroformate, isocyanate, PNP, tosylate, N-HOBT, and N-hydroxysuccinimidyl.

In one aspect, the activated PEG can include, but not limited to, methoxypolyethylene glycol-succinate, methoxypolyethylene glycol-succinimidyl succinate (mPEG-NHS), methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the conjugates described herein. Methods of preparing polymers having terminal carboxylic acids in high purity are described in U.S. Pat. No. 7,989,554, the content of which is incorporated herein by reference.

In alternative aspects, polymers having terminal amine groups can be employed to make the conjugates described herein. The methods of preparing polymers containing terminal amines in high purity are described in U.S. Pat. Nos. 7,868,131 and 7,569,657, the contents of each of which are incorporated by reference.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further aspect and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will appreciate realize that the foregoing list is merely illustrative and that all polymeric materials having the qualities described herein are contemplated. For purposes of the present invention, “substantially or effectively non-antigenic” means polymeric materials understood in the art as being nontoxic and not eliciting an appreciable immunogenic response in mammals.

In a further aspect of the invention, there is provided the method as described above where the activated polymer is an activated polyethylene glycol (PEG). In another aspect of the invention, the activated PEG includes a leaving group selected from the group consisting of N-hydroxybenzotriazolyl, halogen, N-hydroxy-phthalimidyl, p-nitrophenoxy, imidazolyl, N-hydroxysuccinimidyl; thiazolidinyl thione, O-acyl ureas or

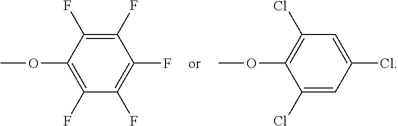

In a certain embodiment, there is provided the method as described above where the activated PEG is selected from the group consisting of:

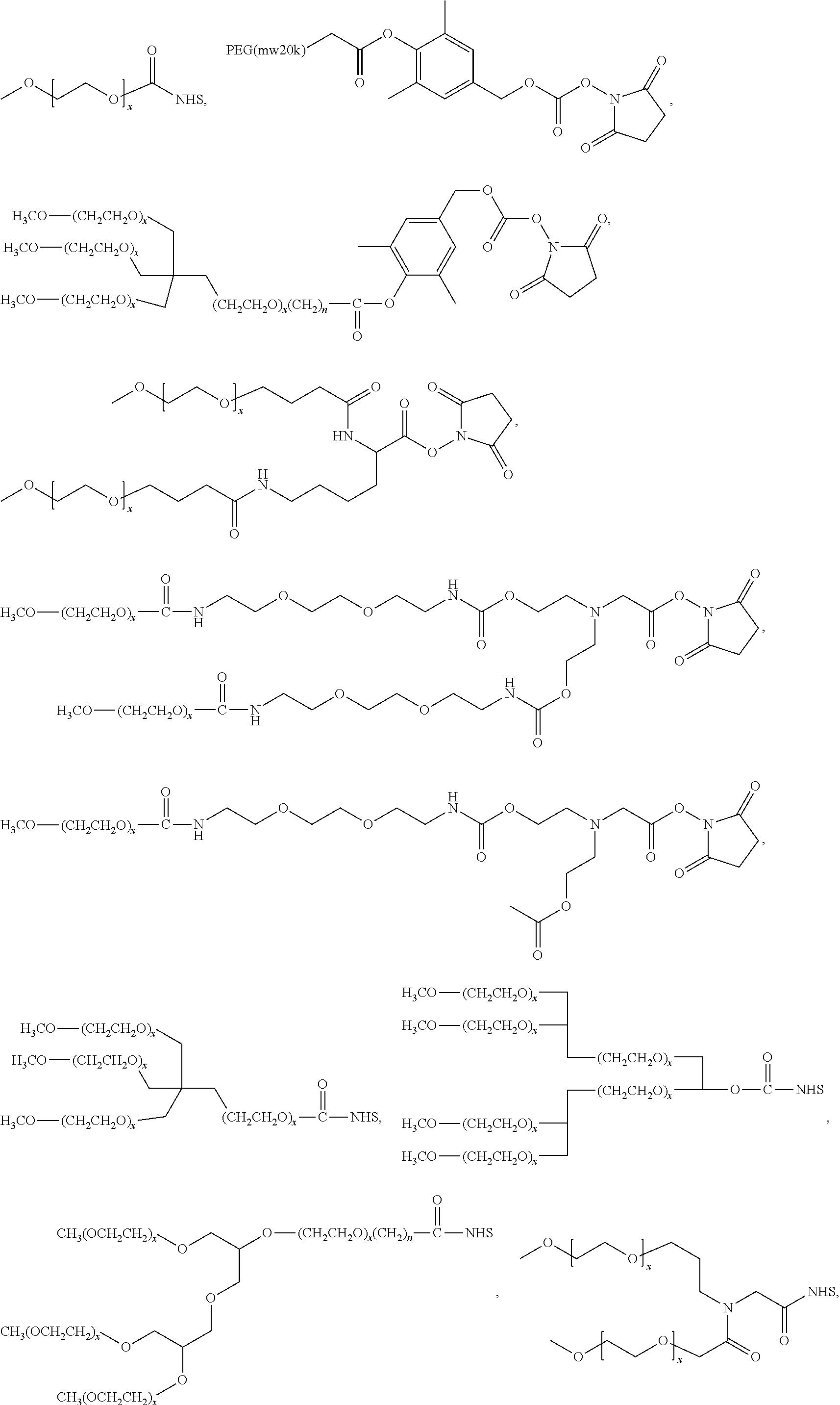

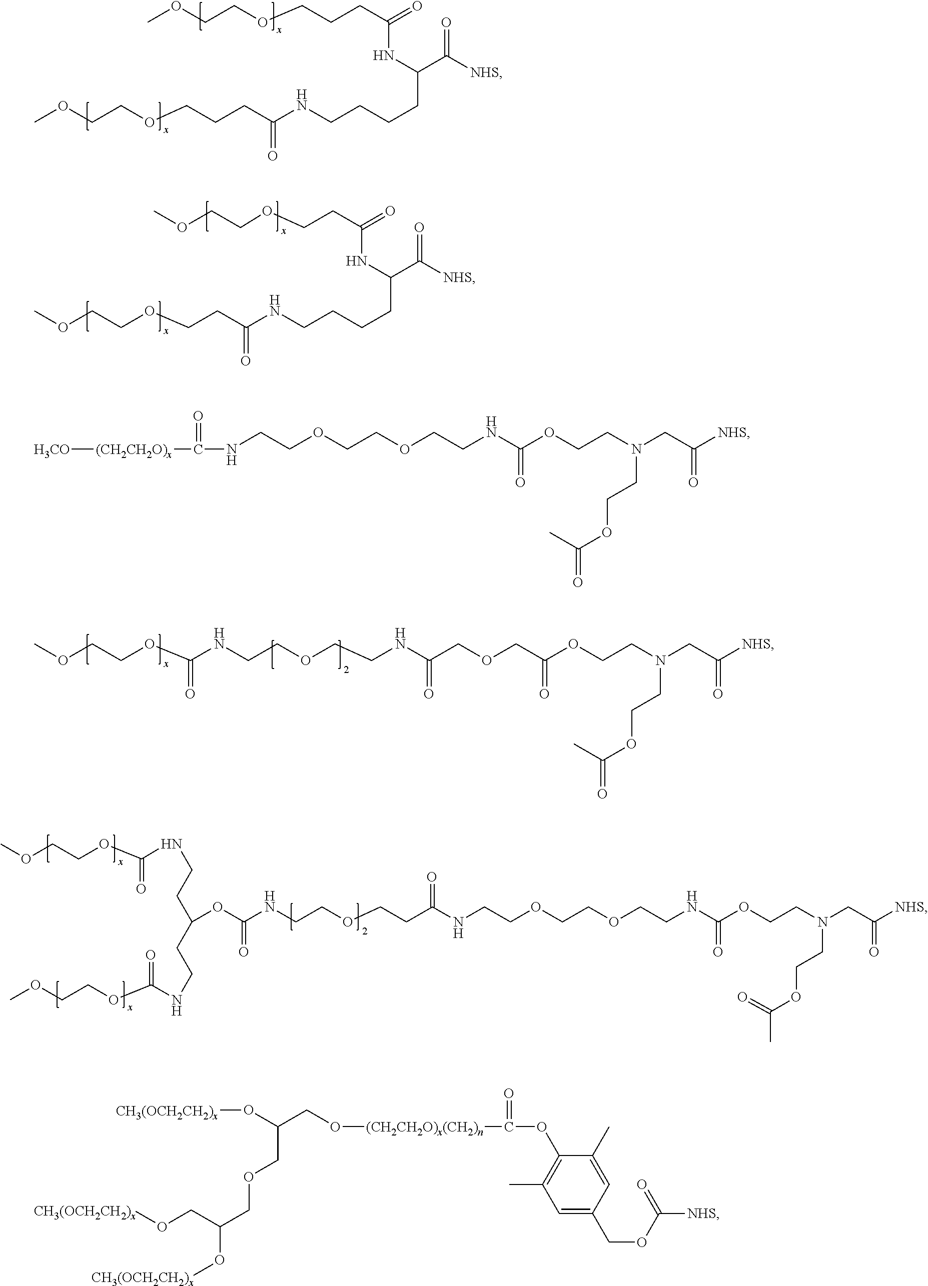

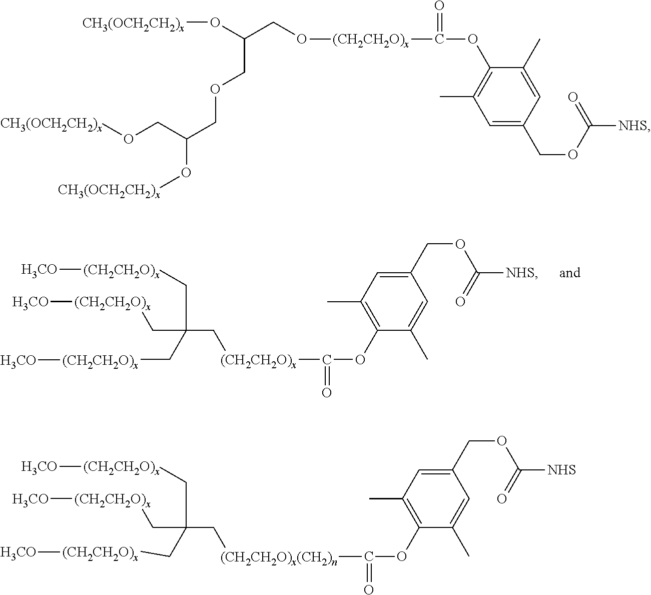

wherein, NHS is

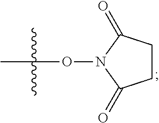 [SMILES: *ON1C(=O)CCC1=O]

(x) is a degree of polymerization positive integer of from about 10 to about 2,300 so that the polymeric portion of the compound has the total number average molecular weight of from about 2,000 to about 100,000 daltons; and

(n) is an integer selected from the group consisting of 1, 2, 3, 4, 5, 6, and 7.

In another embodiment, the activated PEG comprises:

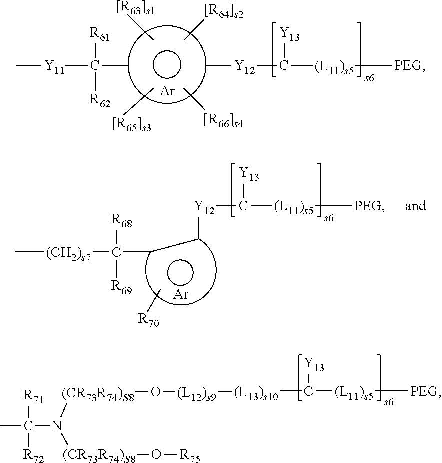

wherein

PEG is a polyethylene glycol;

Y₁₁ is O, or S;

Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;

Y₁₃ is O, S, or NR₆₇;

L₁₁₋₁₃ are independently bifunctional linking moiety selected from the group consisting of
—[C(═O)]ₛ₁₁CR₇₆R₇₇OCR₇₆R₇₇[C(═O)]ₛ₁₂—[Y₁₅]ₛ₁₃—;
—[C(═O)]ₛ₁₁CR₇₆R₇₇NR₇₈CR₇₆R₇₇[C(═O)]ₛ₁₂—[Y₁₅]ₛ₁₃—;
—[C(═O)]ₛ₁₁CR₇₆R₇₇SCR₇₆R₇₇[C(═O)]ₛ₁₂—[Y₁₅]ₛ₁₃—; and
—[C(═O)]ₛ₁₁(CR₇₆R₇₇)ₛ₁₁[C(═O)]ₛ₁₂—[Y₁₅]ₛ₁₃—;
or C(═Y₁₃)-L11- together form an amino acid;

L₁₄ is a bifunctional linking moiety, and the same as defined as L₁ and L₂;

R₆₁, R₆₂, R₆₇, R₇₁, R₇₂, R₇₃ and R₇₄ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;

R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;

R₇₅ is H, —C(═O)—R₇₉, wherein R₇₉, in each occurrence, is the same or different alkyl,

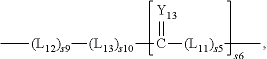

or
a targeting group;

R₇₆, R₇₇ and R₇₈ are independently selected from the group consisting of from H, C₁₋₆ alkyl, C₂₋6 alkenyl, C₂₋₆ alkynyl, C₁₋₆ heteroalkyl and aryl;

Ar is a moiety which when included in the formula forms an aromatic or heteroaromatic hydrocarbon;

(s1), (s2), (s3), and (s4) are independently zero or one;

(s5) is a positive integer of from about 1 to about 6;

(s6) is zero or a positive integer;

(s7) is zero, one or two;

(s8) is 1, 2 or 3;

(s9) is zero or one;

(s10) is zero or a positive integer; and

(s11), (s12), and (s13) are independently zero or one.

In a preferred embodiment, the PEG is mPEG.

In another preferred embodiment, the activated PEG is selected from the group consisting of:

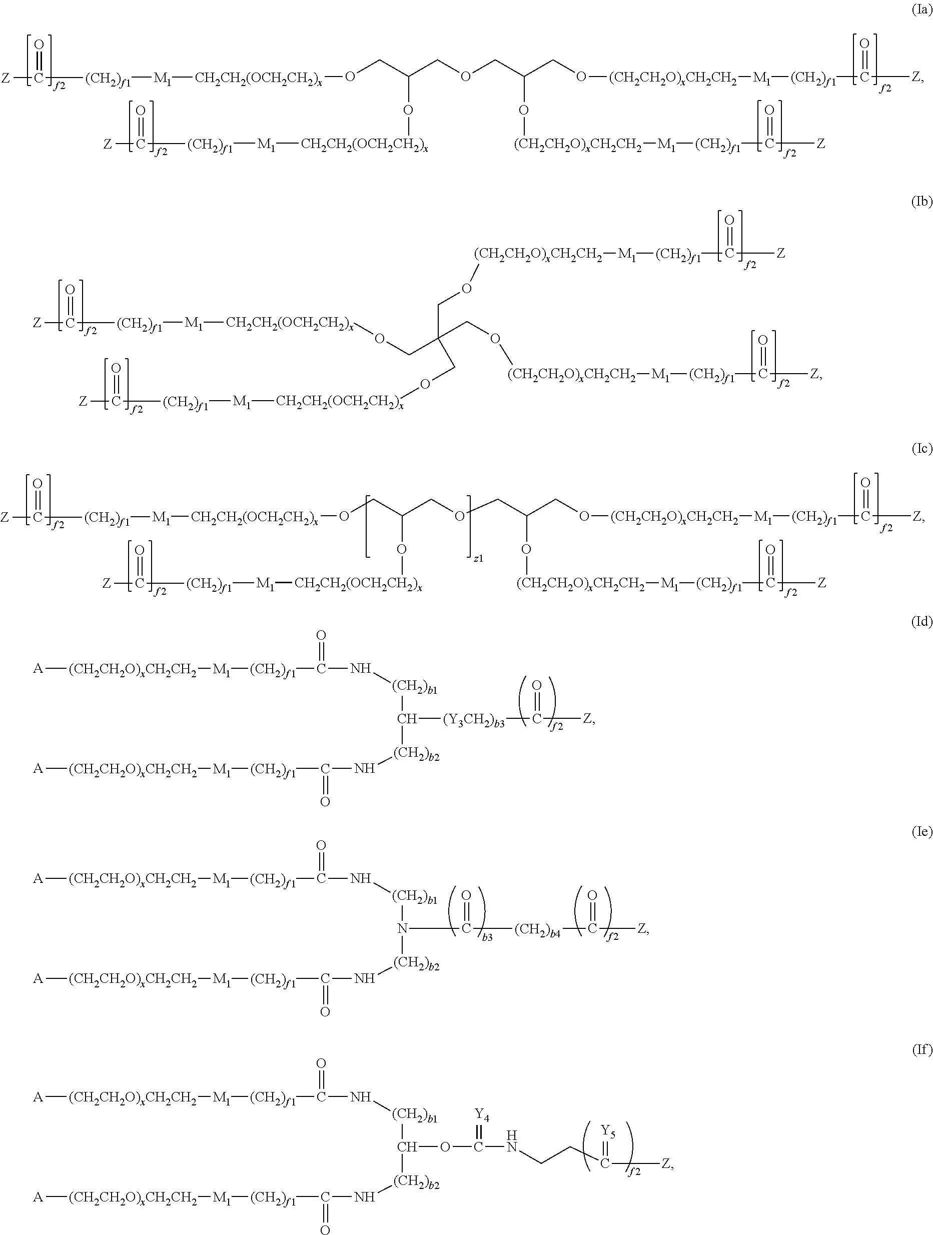

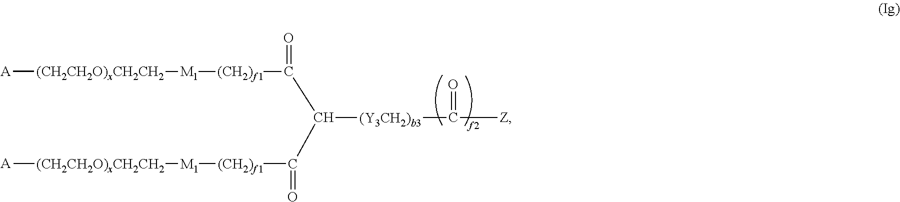

(Ih) Z—[C(═O)]_{f2}—(CH₂)_{f1}-M₁-CH₂CH₂—O—(CH₂CH₂O)ₓ—CH₂CH₂-M₁-(CH₂)_{f1}—[C(═O)]_{f2}—Z, and

(Ii) A-(CH₂CH₂O)ₓ—CH₂CH₂-M₁-(CH₂)_{f1}-[C(═O)]_{f2}—Z,
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;

R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, an activating group;

(b1) and (b2) are independently zero or positive integers;

(b3) is zero or 1;

(b4) is a positive integer;

(f1) is zero or a positive integer of from about 1 to about 10;

(f2) is zero or 1;

(z1) is zero or a positive integer of from 1 to about 27;

(x) is a degree of polymerization positive integer of from about 10 to about 2,300 so that the polymeric portion of the compound has the total number average molecular weight of from about 2,000 to about 100,000 daltons, provided that one or more Z are a leaving group.

In a certain embodiment, the molecular weight of the polymer ranges from about 2,000 to about 60,000 daltons, preferably the molecular weight of the polymer ranges from about 5,000 to about 50,000 daltons, and more preferably from about 20,000 to about 40,000 daltons.

In another embodiment, the polymer is conjugated via a linker. In yet another embodiment, the polymer is conjugated via amine, amide bond or carbamate bond, releasable or permanent.

### Synthesis of Conjugates

Generally, the polymer target conjugate which is substantially free of polymer attachment on the N-terminal of said target, said target being selected from the group consisting of proteins, peptides, and the like conjugates described herein are prepared by (a) reacting a target with a capping reagent under conditions to selectively cap the N-terminal of the target; (b) reacting the N-terminal-capped target with an activated polymer under conditions to allow polymer attachment to at least one site on the N-terminal-capped target to form a polymer-target conjugate which is substantially free of polymer attachment on the N-terminal of said target.

The reaction of the target with the capping reagent is preferably carried out under conditions which include an aqueous environment in which the pH is about 4.0 to about 7.0. If desired, the capping group attached to the N-terminal can be reduced with a reducing agent to form a permanently attached cap thereon.

Suitable reducing agents include, for example, sodium cyanoborohydride (NaBH₃CN), sodium triacetoxyborohydride (NaBH(OC(═O)OCH₃)₃), sodium hydride, decaborane (B₁₀H₁₄), InCl₃-Et₃SiH complex, Nickel nanoparticles, Et₃SiH-iridium complex, and Ti(iOPr)₄. One preferable reducing agent is sodium cyanoborohydride.

In another aspect of the invention, the N-terminal, C-terminal or both terminals of the target is capped with a permanent or transitional group for further reaction of Lysine or Histidine to react with an activated polymer such as an activated polyalkylene oxide, or PEG. Furthermore, the capping group on N- and/or C-terminal of the PEGylated target is removed to restore the natural amine and carboxylic functional group. In certain instances, the N- and/or C-terminal of the target is required to express its biological efficacy or to bind to the corresponding receptors.

In a certain embodiment, there is provided the method as described above wherein the conditions to allow polymer attachment include reacting the activated polymer with the N-terminal capped target in the presence of an aqueous buffer having a pH of from about 6.0 to about 10.0. Preferably, the pH is from about 7.0 to about 10.0.

In a further embodiment, the polymer is attached to an epsilon amino group of a lysine. In an alternative embodiment, the polymer is attached to a histidine.

In one embodiment, the activating group is an aldehyde and the reaction is carried out in the presence of a reducing agent.

As will be appreciated by those of ordinary skill, the aldehyde derivatives are used for N-terminal attachment of the polymer to the target, such as IFN-gamma. For example, polyalkylene oxide (PAO) aldehydes react preferably with amines and undergo reductive amination in the presence of sodium cyanoborohydride to form a secondary or tertiary amine. Suitable polyethylene glycol (PEG) aldehydes are available from NOF and other commercial sources. Alternatively, the aldehyde can react with epsilon amine of lysine or histidine in IFN-gamma or the secondary amine of histidine to form a tertiary amine.

In other aspects of the invention, the other activated linkers shown above will allow for non-specific linkage of the polymer to Lys amino groups-forming carbamate (urethane) or amide linkages.

In another embodiment, the activating group is selected from the group consisting of carbonyl imidazole, chloroformate, isocyanate, PNP, tosylate, N-HOBT, and N-hydroxysuccinimidyl.

In some aspects of the invention, the activating group for the polymer is an oxycarbonyl-oxy-N-dicarboximide group such as a succinimidyl carbonate group. Alternative activating groups include N-succinimide, N-phthalimide, N-glutarimide, N-tetrahydrophthalimide and N-norborene-2,3-dicarboxide. These urethane-forming groups are described in commonly owned U.S. Pat. No. 5,122,614, the disclosure of which is hereby incorporated by reference. Other urethane-forming activated polymers such as benzotriazole carbonate activated (BTG-activated PEG-available from Nektar) can also be used. See also commonly-assigned U.S. Pat. No. 5,349,001 with regard to the above-mentioned T-PEG.

For purposes of illustration, suitable conjugation reactions include reacting a target, such as IFN-gamma, with a suitably activated polymer system described herein. The reaction is preferably carried out using conditions well known to those of ordinary skill for protein modification, including the use of a PBS buffered system, etc. with the pH in the range of about 5.0-5.5. It is contemplated that in most instances, an excess of the activated polymer will be reacted with the target.

Reactions of this sort will often result in the formation of conjugates containing one or more polymers attached to the target. As will be appreciated, it will often be desirable to isolate the various fractions and to provide a more homogenous product. In most aspects of the invention, the reaction mixture is collected, loaded onto a suitable column resin and the desired fractions are sequentially eluted off with increasing levels of buffer. Fractions are analyzed by suitable analytical tools to determine the purity of the conjugated protein before being processed further.

It will also be appreciated that heterobifunctional polyalkylene oxides are also contemplated for purposes of cross-linking the target, or providing a means for attaching other moieties such as targeting agents for conveniently detecting or localizing the polymer target conjugate in a particular areas for assays, research or diagnostic purposes.

In a certain embodiment, the polymer-target conjugate of the present invention is selected from among:

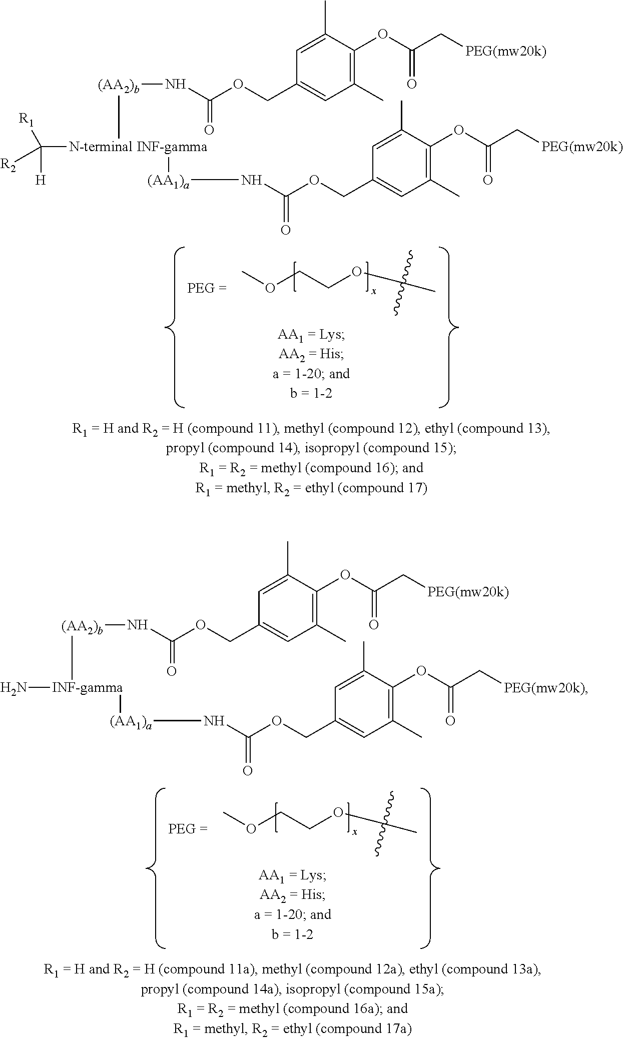

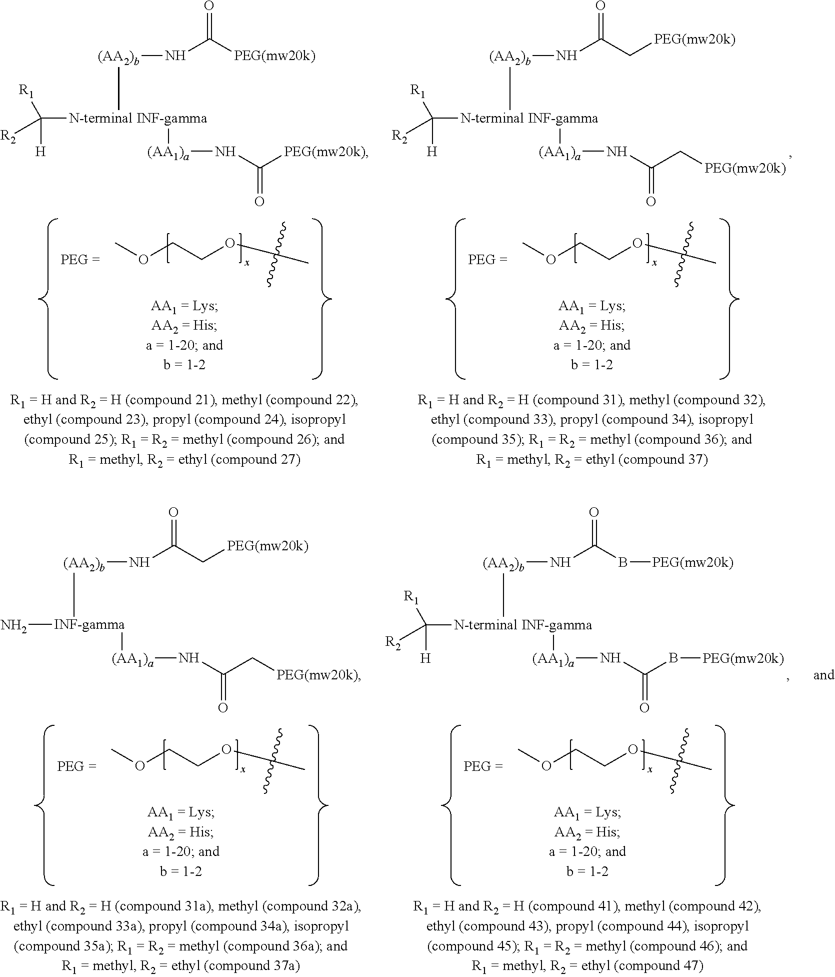

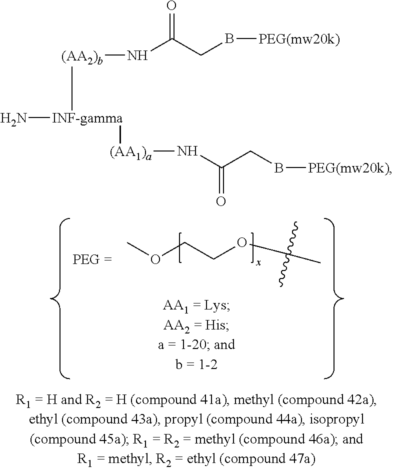

wherein,
(x) is a degree of polymerization positive integer of from about 10 to about 2,300 so that the polymeric portion of the compound has the total number average molecular weight of from about 2,000 to about 100,000 daltons;
B-PEG is selected from the group consisting of:

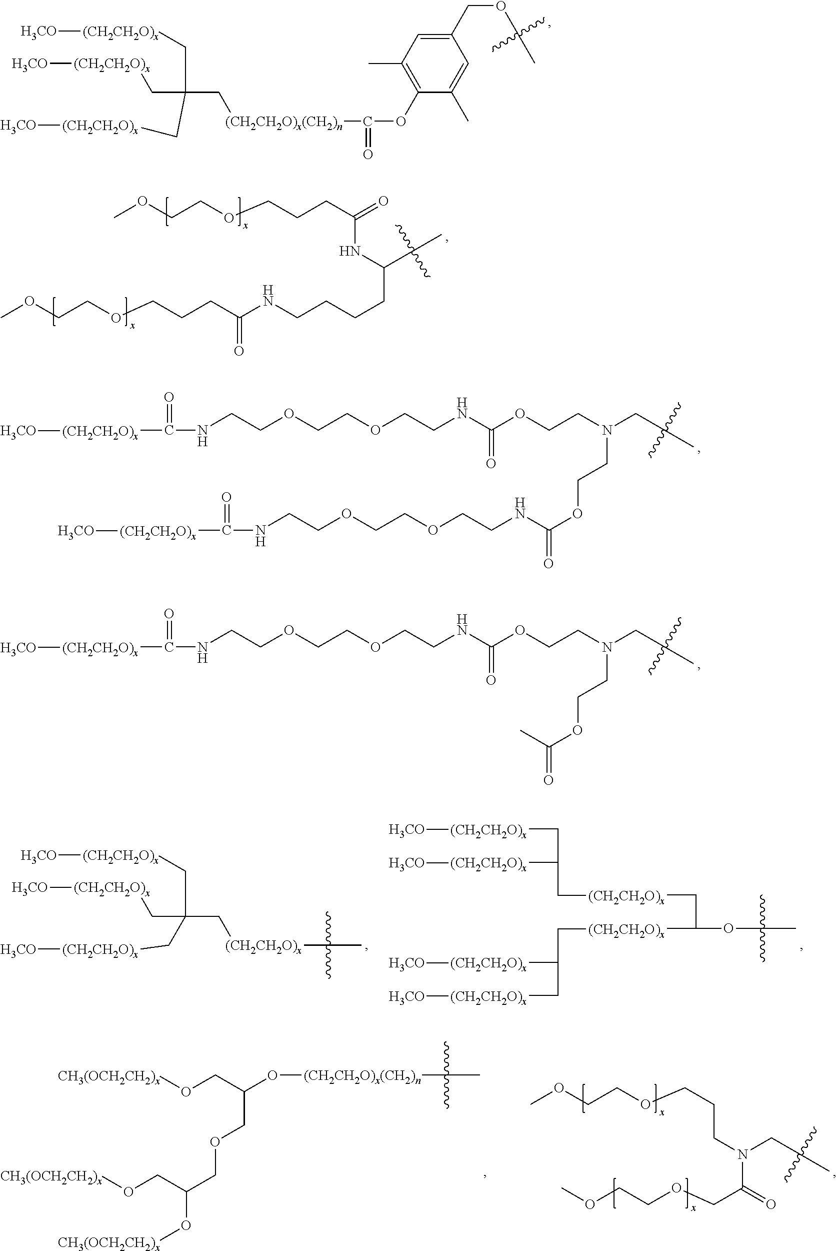

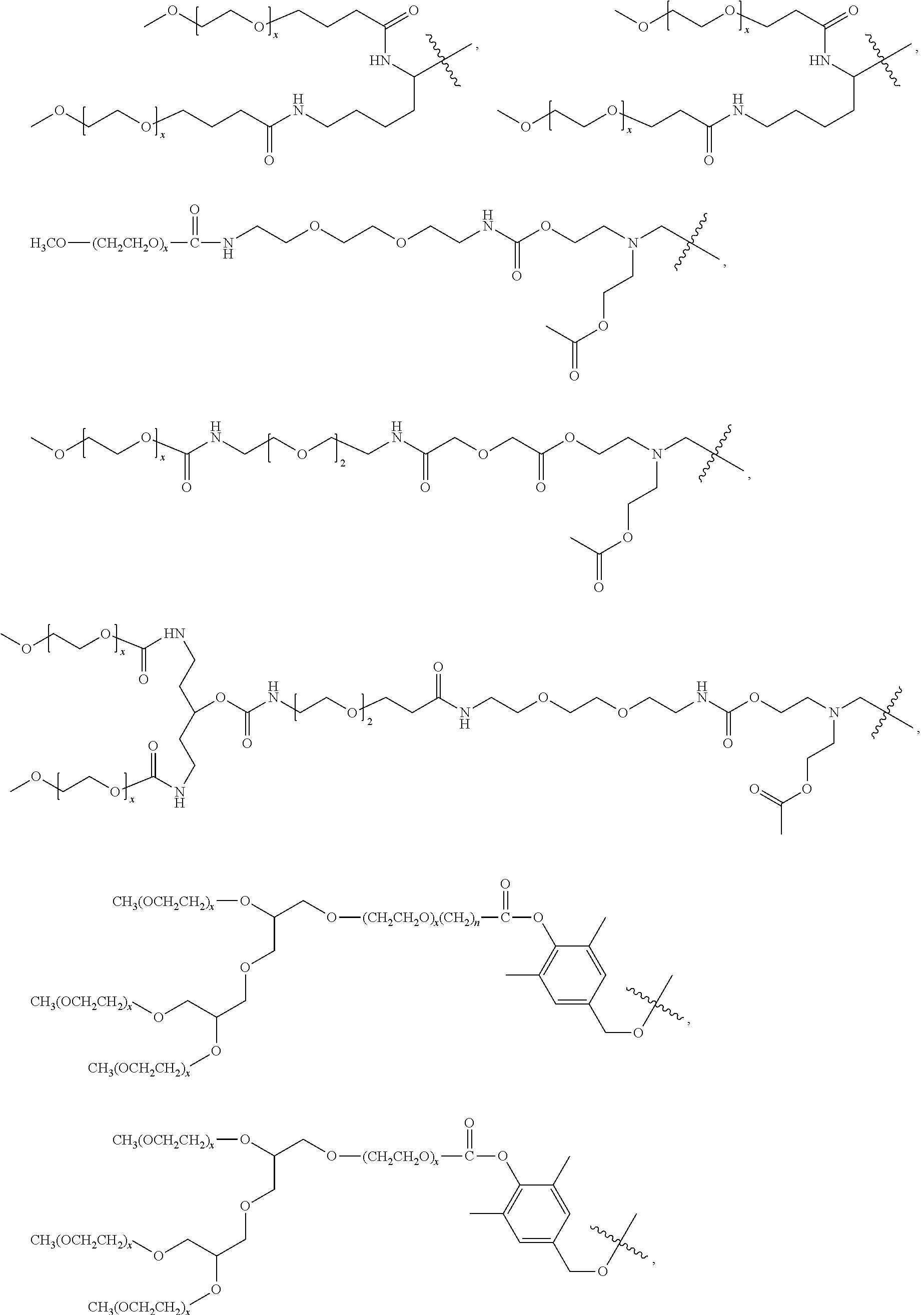

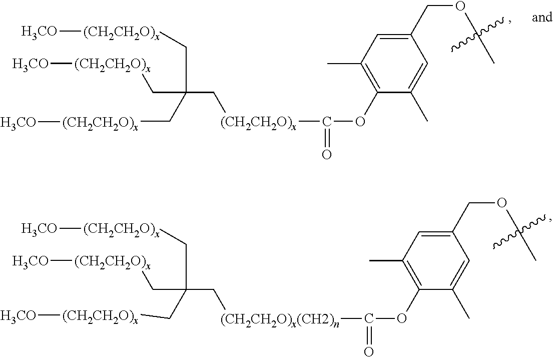

wherein all variables are as previously defined.

### Polymer Target Conjugates

In certain aspects of the invention there are provided polymer target conjugates in which a target moiety such as described above, e.g., polypeptides, proteins and the like, having at least one polymer attached thereto at a site which is not the N-terminal of the target.

The invention also includes compositions containing a plurality of polymer-polypeptide conjugates. The polymer-polypeptide conjugates have a polypeptide covalently attached to at least one polymer through an epsilon amino group of a Lysine or a Histidine found on the polypeptide. The conjugates also preferably contain less than 5%, and more preferably less than 1%, of the polymer-polypeptide conjugates having a polymer attached to the N-terminal of the polypeptide. Within this aspect of the invention, some preferred polymer target conjugates interferon-gamma-PEG conjugates.

Optionally, the N-terminal includes a capping group covalently attached to the N-terminal of the target moiety.

### DEFINITIONS

For purposes of the present invention, the term “residue” shall be understood to mean that portion of a conjugate, to which it refers, e.g., amino acid, etc. that remains after it has undergone a substitution reaction with another conjugate.

For purposes of the present invention, the term “polymeric containing residue” or “PEG residue” shall each be understood to mean that portion of the polymer or PEG which remains after it has undergone a reaction with a target, such as an interferon.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or conjugate with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

The terms “effective amounts” and “sufficient amounts” for purposes of the present invention shall mean an amount which achieves a desired effect or therapeutic effect as such effect is understood by those of ordinary skill in the art.

### EXAMPLES

The following examples serve to provide further appreciation of the invention but are not meant in any way to restrict the effective scope of the invention.

### Example 1

### Preparation of N-terminal protected IFN-gamma (compounds 1-7)

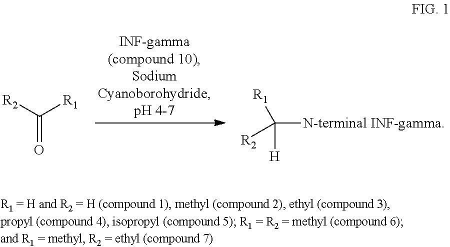

Interferon-gamma (IFN-gamma) is suspended in a 100 mM sodium acetate buffer at pH 5.0-5.5 in 1.5 mg/ml concentration. To the suspension, a ketone or aldehyde containing compound, R₁—C(═O)—R₂, is added at 10-15 times excess molar ratio to IFN-gamma in the presence of sodium cyanoborohydride. The concentration of sodium cyanoborohydride is kept at 15 mM and the reaction is conducted at 20° C. for 16 hours to provide the product.

### Example 2

### Preparation of N-terminal protected IFN-gamma (compound 8)

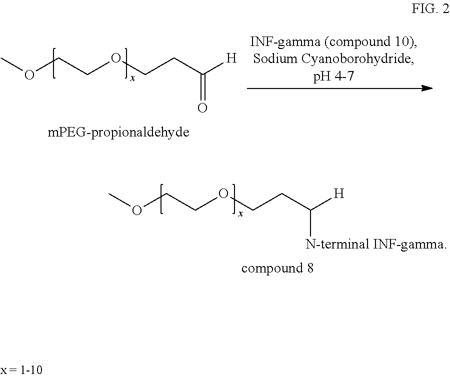

IFN-gamma is reacted with oligoalkylene oxide (e.g. mPEG-propyl aldehyde) under the same conditions as provided in Example 1 to provide the product.

### Example 3

### Preparation of N-terminal protected IFN-gamma (compounds 1a-7a)

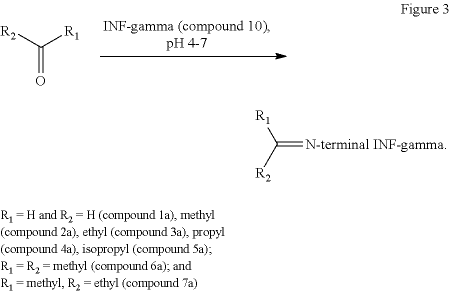

Interferon-gamma (IFN-gamma) is suspended in a 100 mM sodium acetate buffer at pH 5.0-5.5 in 1.5 mg/ml concentration. To the suspension, a ketone or aldehyde containing compound, R₁—C(═O)—R₂, is added at 10-15 times excess molar ratio to IFN-gamma. The reaction is conducted at 20° C. for 16 hours to provide the product.

### Example 4

### Preparation of N-terminal protected IFN-gamma (compound 8a)

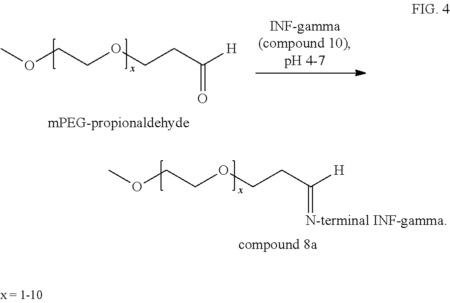

IFN-gamma is reacted with oligoalkylene oxide (e.g. mPEG-propyl aldehyde) under the same conditions as provided in Example 3 to provide the product.

### Example 5

### Preparation of Lys or His PEGylated IFN-gamma (compounds 11-17)

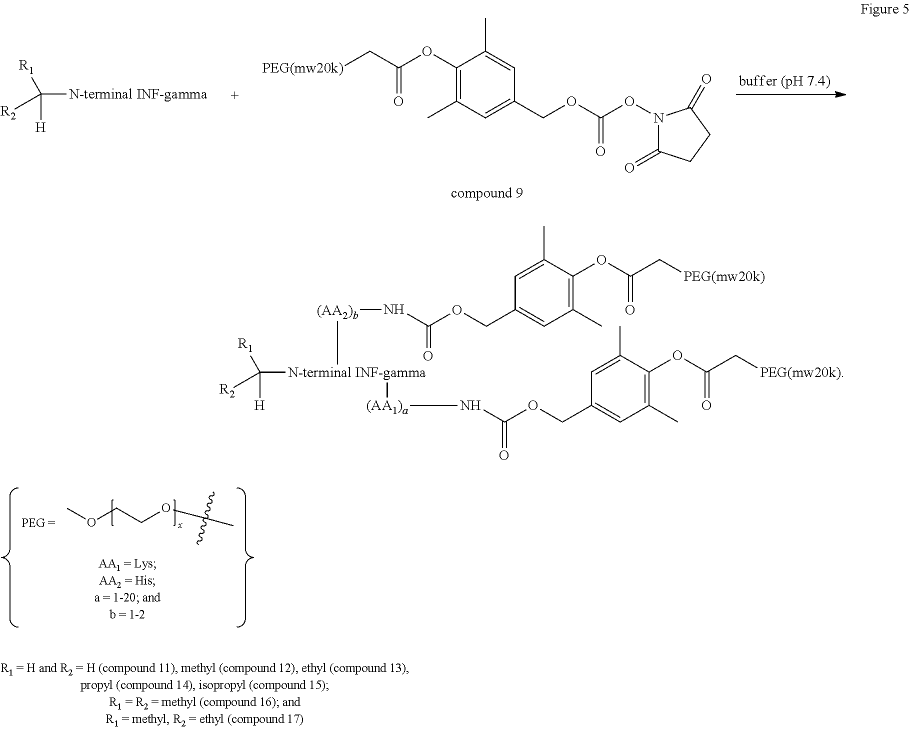

Compound 1, 2, 3, 4, 5, 6, or 7, N-terminal alkylated IFN-gamma, has two histidines and 20 lysine residues. With fast stirring, a 10-15 molar excess of an activated PEG powder, compound 9, is added to a solution of N-terminal alkylated IFN-gamma, 5 mg/mL in 0.1 M phosphate buffer, pH 7.4. After stirring for 45 min at 25° C., the reaction is treated with 0.2 M sodium phosphate (pH 5.1) to a final pH of 6.5. The reaction mixture is dialyzed against 20 mM sodium phosphate, pH 5.1, at 4° C., using 6,000-8,000 MW cutoff membrane to provide the product.

### Example 6

### Preparation of Lys or His rPEGylated IFN-gamma (compound 18)

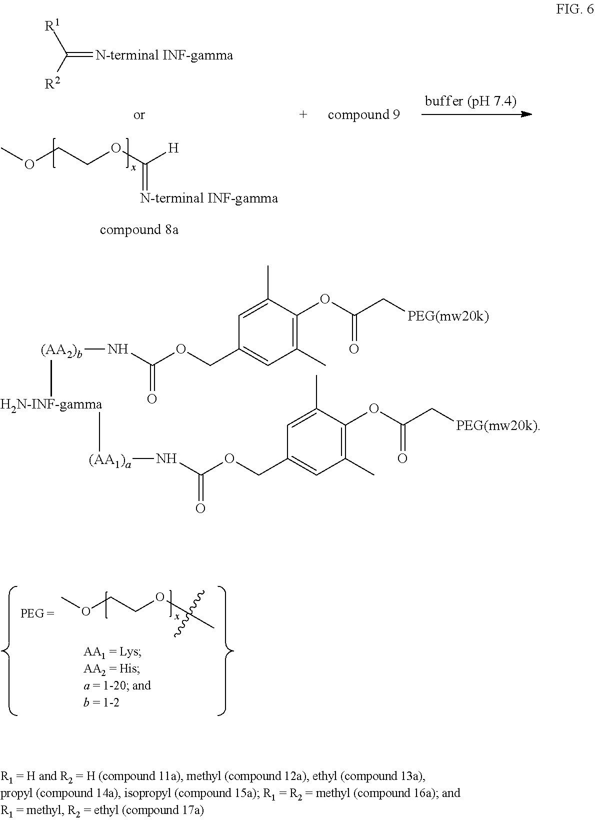

Compound 1a, 2a, 3a, 4a, 5a, 6a, 7a, or 8a is reacted with compound 9 at pH 7.4 in the same condition as described in Example 5 to provide the product.

### Example 7

### Preparation of Lys or His scPEGylated IFN-gamma (compounds 21-27)

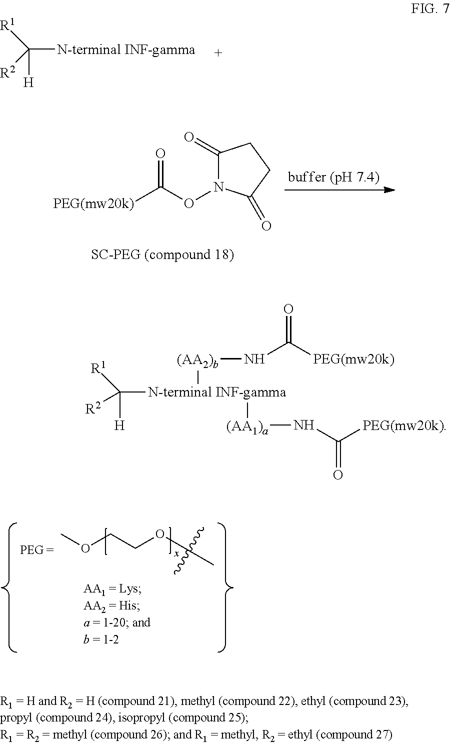

Compound 1, 2, 3, 4, 5, 6, or 7, N-terminal alkylated IFN-gamma, is reacted with compound 18 at pH 7.4 in the same condition as described in Example 5 to provide the product.

### Example 8

### Preparation of Lys or His scPEGylated IFN-gamma (compounds 21a-27a)

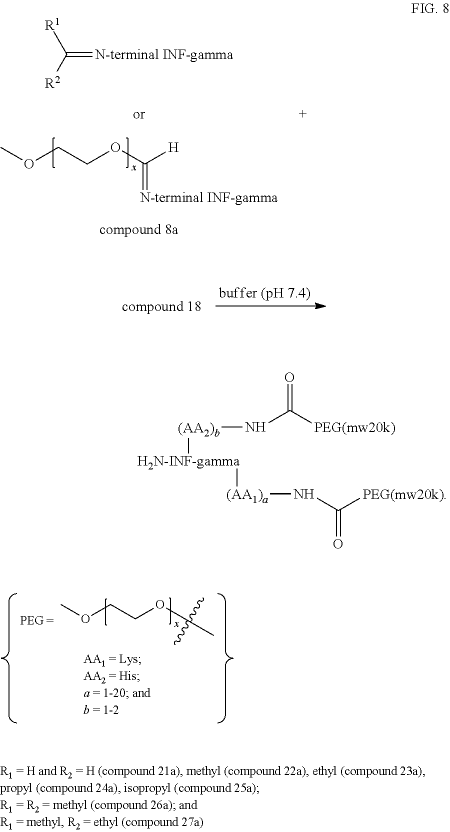

Compound 1a, 2a, 3a, 4a, 5a, 6a, 7a, or 8a is reacted with compound 18 at pH 7.4 in the same condition as described in Example 5 to provide the product.

### Example 9

### Preparation of Lys or His tPEGylated IFN-gamma (compounds 31-37)

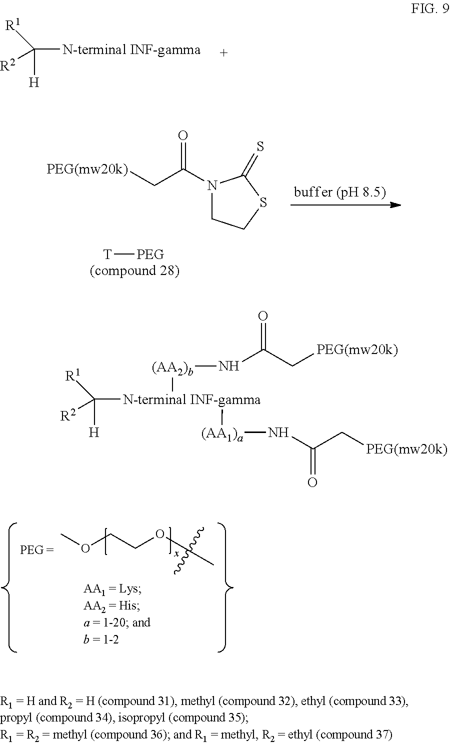

Compound 1, 2, 3, 4, 5, 6, or 7, N-terminal alkylated IFN-gamma, is reacted with compound 28 at pH 8.5 in the same condition as described in Example 5 to provide the product.

### Example 10

### Preparation of Lys or His tPEGylated IFN-gamma (compounds 31a-37a)

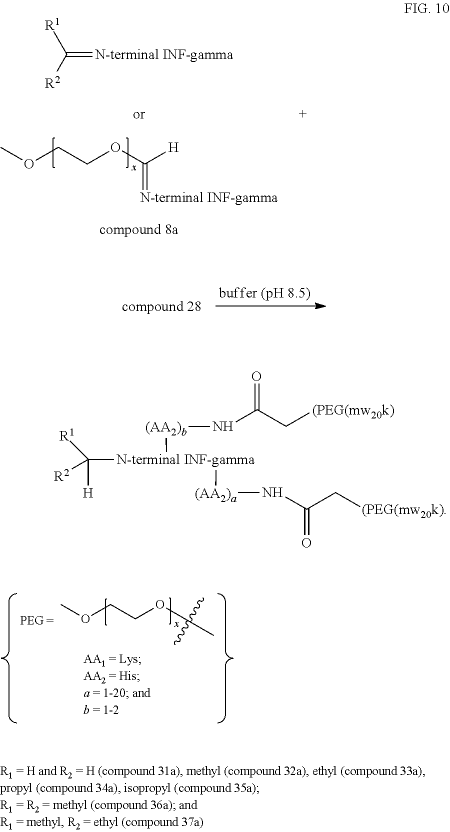

Compound 1a, 2a, 3a, 4a, 5a, 6a, 7a, or 8a is reacted with compound 18 at pH 8.5 in the same condition as described in Example 5 to provide the product.

### Example 11

### Preparation of Lys or His B-PEGylated IFN-gamma (compounds 41-47)

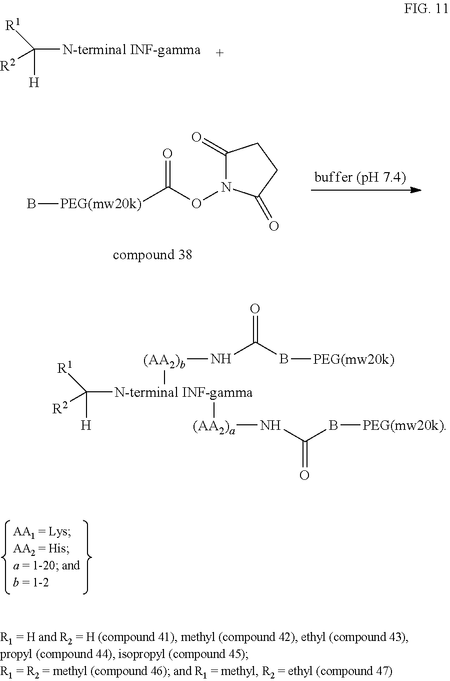

Compound 1, 2, 3, 4, 5, 6, or 7, N-terminal alkylated IFN-gamma, is reacted with compound 28 at pH 7.4 in the same condition as described in Example 5 to provide the product.

### Example 12

### Preparation of Lys or His B-PEGylated IFN-gamma (compounds 41a-47a)

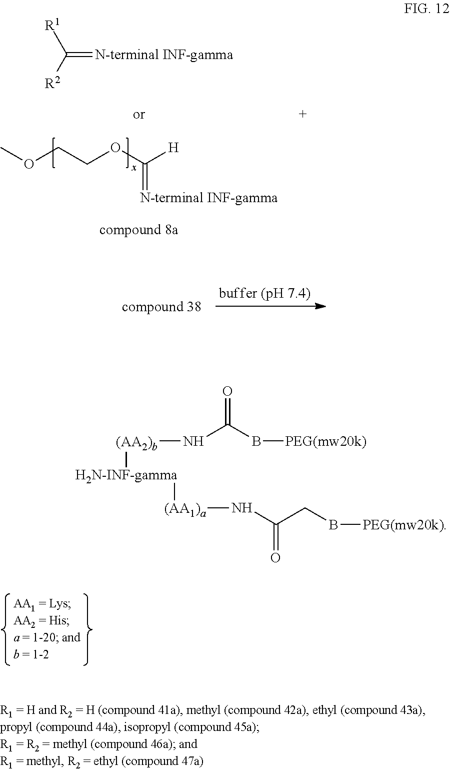

Compound 1a, 2a, 3a, 4a, 5a, 6a, 7a, or 8a is reacted with compound 18 at pH 7.4 in the same condition as described in Example 5 to provide the product.

### Example 13

### Purification of PEGylated Product

PEGylated IFN-gamma is purified by weak anion exchange column (HiTrap DEAE FF, 1 ml. GE Healthcare) or by hydrophobic interaction column (HIC phenyl FF, 1 ml. GE Healthcare). In DEAE column purification, Buffer A contained 10 mM Tris, pH 8.5 and buffer B had 0.5 M NaCl in buffer A. Elution is conducted at 1 ml/min over 30 min. In HIC phenyl purification, Buffer A contained 0.75 M ammonium sulfate in PBS buffer and buffer is PBS. Elution is conducted at 1 ml/min over 30 min. The less PEGylated IFN-gamma elutes earlier than more PEGylated IFN-gamma. Each isolated fractions is combined based on its composition and the combined fraction is concentrated using Centricon YM30 and buffer-exchanged to PBS by NAP-5 column to provide the purified product.

### Example 14

### Analysis of Composition of PEGylated IFN-gamma

The concentration of PEG in PEGylated IFN-gamma is determined by UV at 280 nm. 1.5-μg protein is loaded into the gel without sample reduction and heating (Novex NuPAGE 4-12% Bis-Tris gel, Invitrogen). The electrophoresis is conducted at 200 Voltage for 30 min and the protein bands are visualized after simple blue stain. The density of the image is obtained on Molecular Dynamics.

### Example 15

### PEGylated IFN-Gamma Binding Assay

IFN-gamma activity is measured by the binding efficiency to the receptor. Samples, IFN-gamma receptor and a control, are added to 96-well plate, and then IFN-gamma or PEGylated IFN-gamma of the present invention is added. After 10 min incubation at 37° C., the binding efficiency is monitored every 4 minutes kinetically by the change of UV absorption.

The polymer conjugate of the present invention, as measured above, retained significant amount of IFN-gamma binding activity. Without being bound to any theory, it is possible that the present PEG attached to the Lysine or Histidine is still flexible enough to provide freedom for N- and C-terminal of IFN-gamma to form a dimer conformation and to react with the receptor. The above results provide that PEGylation of the present invention did not alter the IFN-gamma biological activity even after the PEGylation.

We claim:

1. A method of preparing a polymer target conjugate which is substantially free of polymer attachment on the N-terminal of said target, said target being selected from the group consisting of proteins, peptides, and the like, comprising:
(a) reacting a target with a capping reagent under conditions to selectively cap the N-terminal of the target;
(b) reacting the N-terminal-capped target with an activated polymer under conditions to allow polymer attachment to at least one site on the N-terminal-capped target to form a polymer-target conjugate which is substantially free of polymer attachment on the N-terminal of said target.

2. The method of claim 1 further comprising isolating the polymer-target conjugate which is substantially free of polymer attachment on the N-terminal of said target resulting from step (b).

3. The method of claim 1 further comprising deprotecting the N-terminal of the polymer-target conjugate.

4. The method of claim 1, wherein said conditions to selectively cap the N-terminal of the target include reacting the target with the capping reagent at pH of from about 4.0 to about 7.0.

5. The method of claim 4, wherein said capping reagent is of the formula
R₁—C(═O)—R₂
wherein R₁ and R₂ are independently selected from the group consisting of H, C₁₋₆ alkyl, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, and 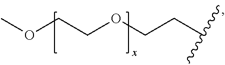
wherein x is an integer selected from the group consisting of 1, 2, 3, 4, 5, 6, 7, 8, 9, and 10.

6. The method of claim 4, wherein said conditions further comprise reducing the bond attaching the capping group to the N-terminal of the target with a reducing agent.

7. The method of claim 1, wherein the target is a protein, a polypeptide, or an interferon.

8. The method of claim 7, wherein said interferon is interferon-gamma (IFN-gamma).

9. The method of claim 9, wherein the interferon-gamma (IFN-gamma) has SEQ ID NO: 1, SEQ ID NO: 2 or SEQ ID NO: 3.

10. The method of claim 8, wherein the polypeptide is erythropoietin (EPO), immunotoxin antibody or a fragment thereof, blood factors.

11. The method of claim 1, wherein the activated polymer is an activated polyethylene glycol (PEG).

12. The method of claim 11, wherein the activated PEG is selected from the group consisting of: 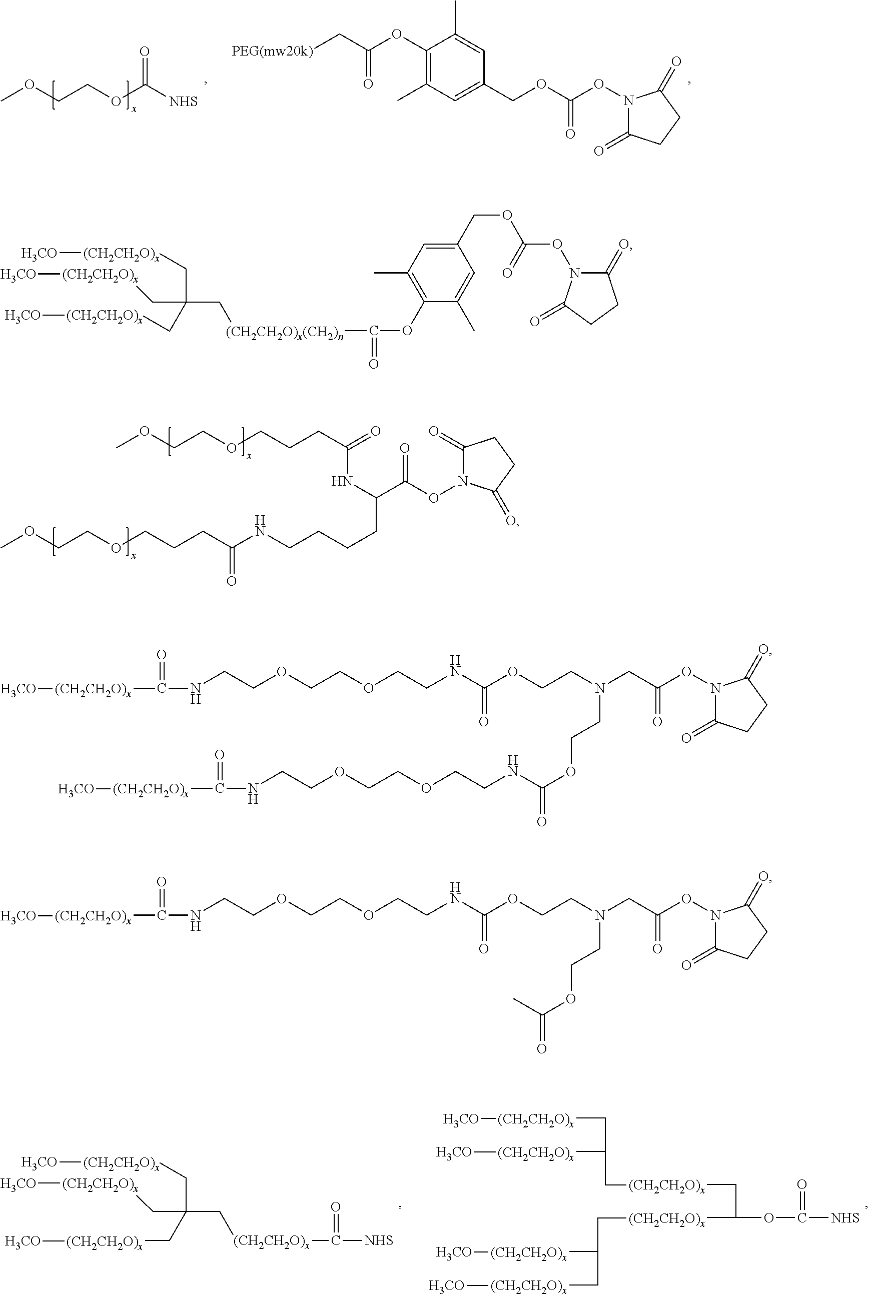 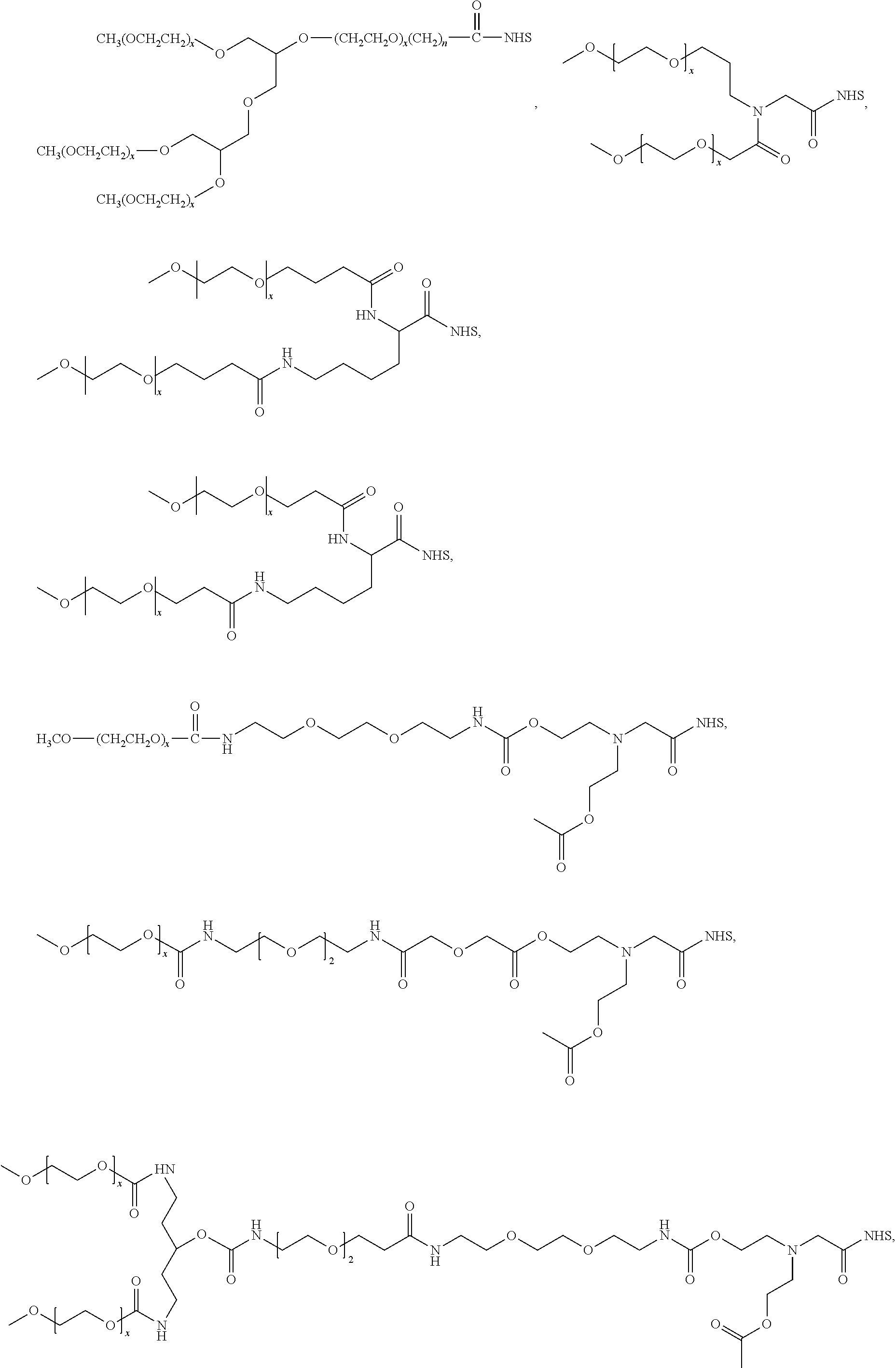 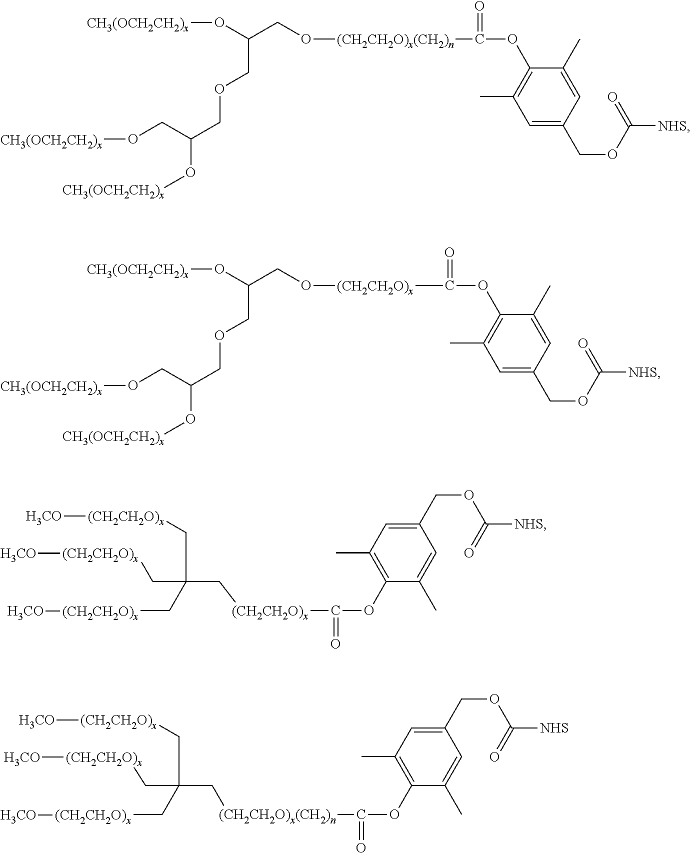
wherein, NHS is 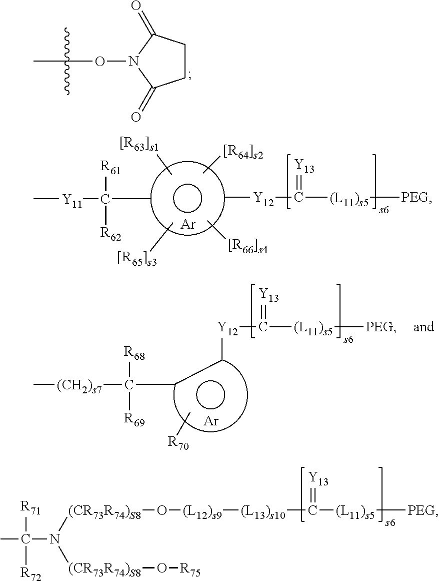
Wherein
(x) is a degree of polymerization positive integer of from about 10 to about 2,300 so that the polymeric portion of the compound has the total number average molecular weight of from about 2,000 to about 100,000 daltons;
(n) is an integer selected from the group consisting of 1, 2, 3, 4, 5, 6, and 7;
PEG is a polyethylene glycol;
Y₁₁ is O, or S;
Y₁₂ is O, S, or NH, provided that L₁₁ is Gly-Phe-Leu-Gly, Ala-Leu-Ala-Leu, Phe-Lys, or Val-Cit, when Y₁₂ is NH and (s6) is a positive integer;
Y₁₃ is O, S, or NR₆₇;
L₁₁₋₁₃ are independently bifunctional linking moiety selected from the group consisting of
—[C(═O)]ₛ₁₁CR₇₆R₇₇OCR₇₆R₇₇[C(═O)]ₛ₁₂—[Y₁₅]ₛ₁₃—;
—[C(═O)]ₛ₁₁CR₇₆R₇₇ NR₇₈CR₇₆R₇₇[C(═O)]ₛ₁₂—[Y₁₅]ₛ₁₃—;
—[C(═O)]ₛ₁₁CR₇₆R₇₇SCR₇₆R₇₇—[C(═O)]ₛ₁₂—[Y₁₅]ₛ₁₃—; and
—[C(═O)]ₛ₁₁(CR₇₆R₇₇)ₛ₁₁[C(═O)]ₛ₁₂—[Y₁₅]ₛ₁₃—;
or C(═Y₁₃)-L11- together form an amino acid;
L₁₄ is a bifunctional linking moiety, and the same as defined as L₁ and L₂;
R₆₁, R₆₂, R₆₇, R₇₁, R₇₂, R₇₃ and R₇₄ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls;
R₆₃, R₆₄, R₆₅ and R₆₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
R₆₈, R₆₉ and R₇₀ are independently selected from the group consisting of C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
R₇₅ is H, —C(═O)—R₇₉, wherein R₇₉, in each occurrence, is the same or different alkyl, 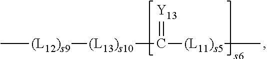
or
a targeting group;
R₇₆, R₇₇ and R₇₈ are independently selected from the group consisting of from H, C₁₋₆ alkyl, C₂₋₆ alkenyl, C₂₋₆ alkynyl, C₁₋₆ heteroalkyl and aryl;
Ar is a moiety which when included in the formula forms an aromatic or heteroaromatic hydrocarbon;
(s1), (s2), (s3), and (s4) are independently zero or one;
(s5) is a positive integer of from about 1 to about 6;
(s6) is zero or a positive integer;
(s7) is zero, one or two;
(s8) is 1, 2 or 3;
(s9) is zero or one;
(s10) is zero or a positive integer; and
(s11), (s12), and (s13) are independently zero or one.

13. The method of claim 14, wherein the activated PEG is mPEG.

14. The method of claim 14, wherein the activated PEG is selected from the group consisting of: 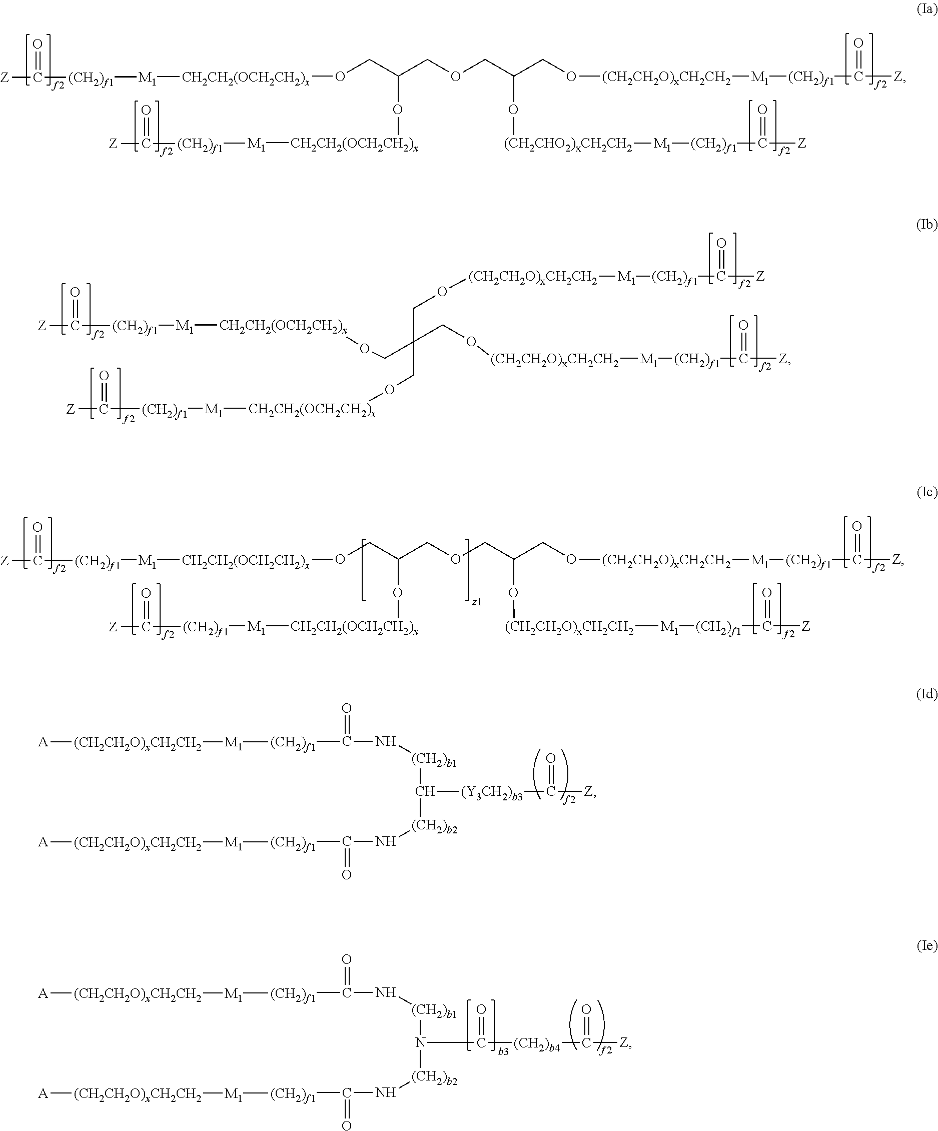 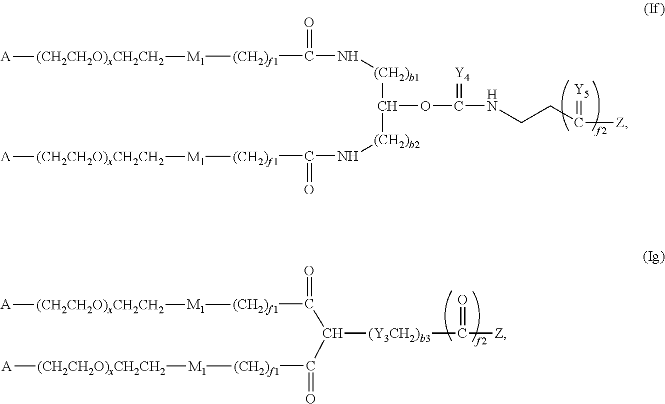
(Ih) Z—[C(═O)]_{f2}—(CH₂)_{f2}-M₁-CH₂CH₂—O—(CH₂CH₂O)ₓ—CH₂CH₂-M₁-(CH₂)_{f1}—[C(═O)]_{f2}—Z, and
(Ii) A-(CH₂CH₂O)ₓ—CH₂CH₂-M₁-(CH₂)_{f1}—[C(═O)]_{f2}—Z,
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, an activating group;
(b1) and (b2) are independently zero or positive integers;
(b3) is zero or 1;
(b4) is a positive integer;
(f1) is zero or a positive integer of from about 1 to about 10;
(f2) is zero or 1;
(z1) is zero or a positive integer of from 1 to about 27;
(x) is a degree of polymerization positive integer of from about 10 to about 2,300 so that the polymeric portion of the compound has the total number average molecular weight of from about 2,000 to about 100,000 daltons, provided that one or more Z is a leaving group.

15. The method of claim 1, wherein the conditions to allow polymer attachment include reacting the activated polymer with the N-terminal capped target in the presence of an aqueous buffer having a pH of from about 6.0 to about 10.0.

16. The method of claim 1, wherein the polymer is attached to an epsilon amino group of a lysine, or the polymer is attached to a histidine.

17. The method of claim 1, wherein the molecular weight of the activated polymer is from about 2,000 to about 100,000 daltons.

18. A polymer target conjugate comprising a target moiety selected from the group consisting of polypeptides, proteins and the like having at least one polymer attached thereto at a site which is not the N-terminal of the target.

19. A composition comprising a plurality of polymer-polypeptide conjugates, said polymer-polypeptide conjugate comprising a polypeptide covalently attached to at least one polymer through an epsilon amino group of a Lysine or a Histidine found on the polypeptide and said conjugates containing less than 5% of the polymer-polypeptide conjugates having a polymer attached to the N-terminal of the polypeptide.

20. The polymer target conjugate of claim 18 selected from the group consisting of: 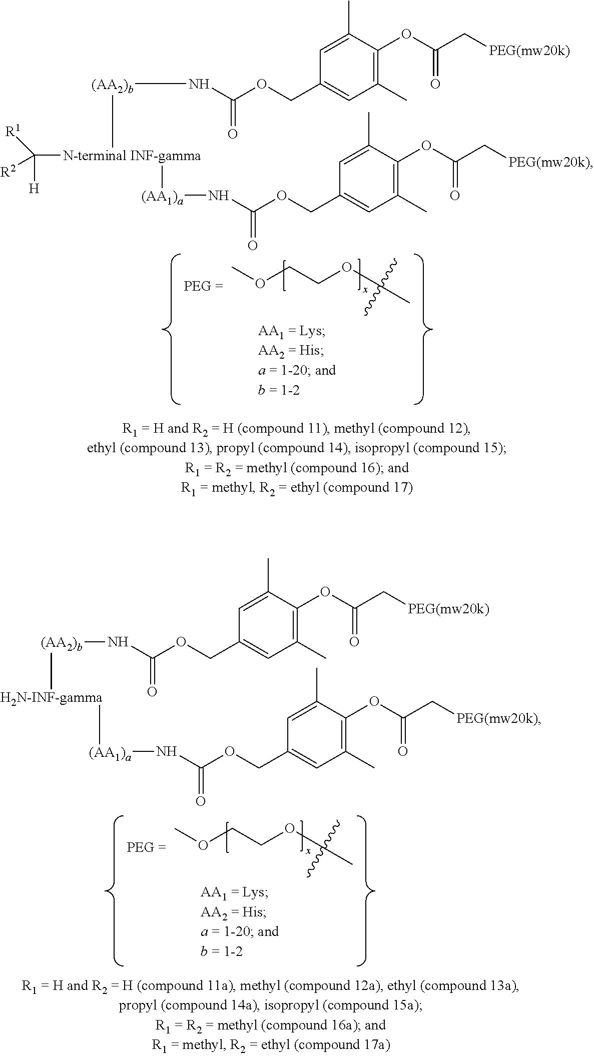 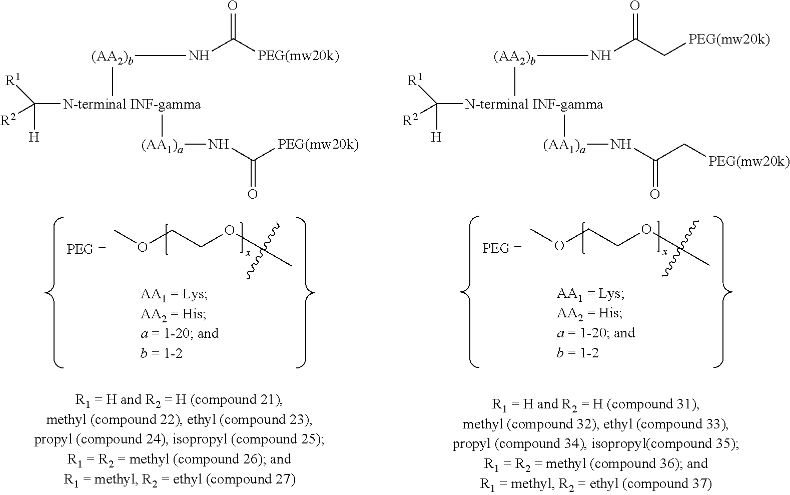 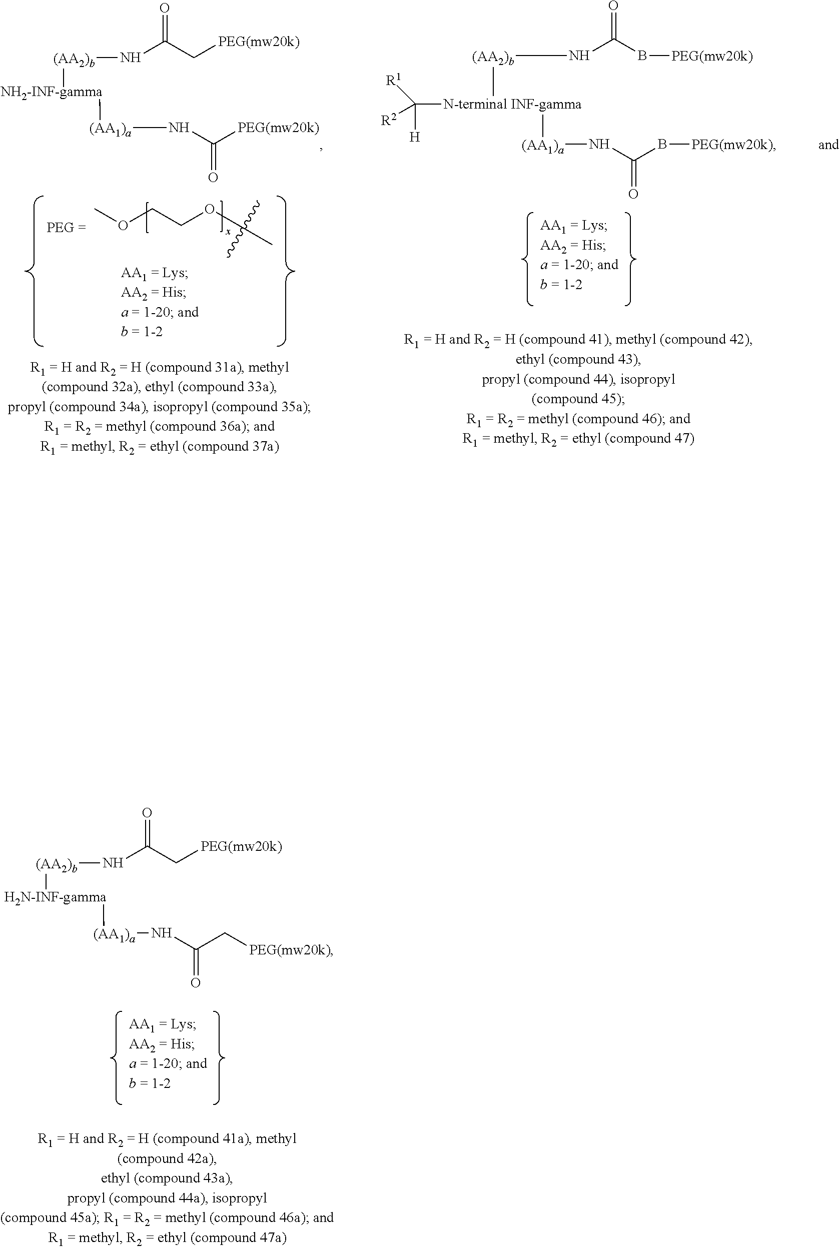
wherein,
(x) is a degree of polymerization positive integer of from about 10 to about 2,300 so that the polymeric portion of the compound has the total number average molecular weight of from about 2,000 to about 100,000 daltons;
B-PEG is selected from the group consisting of: 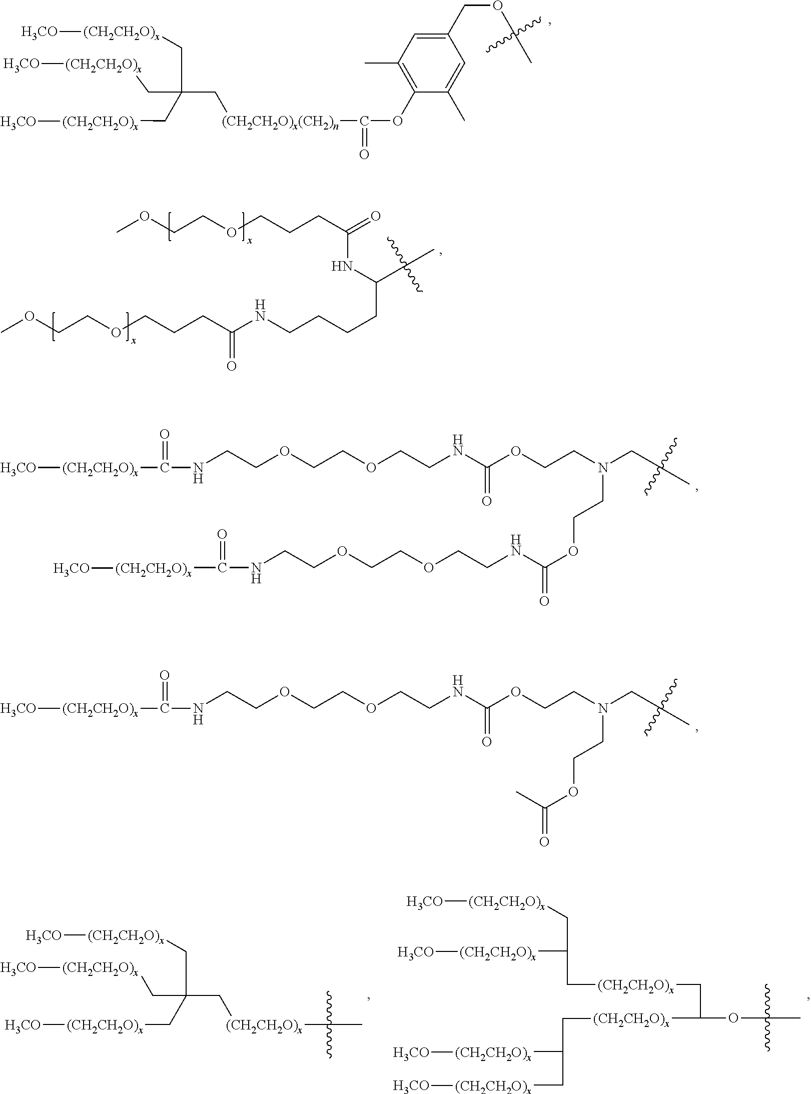 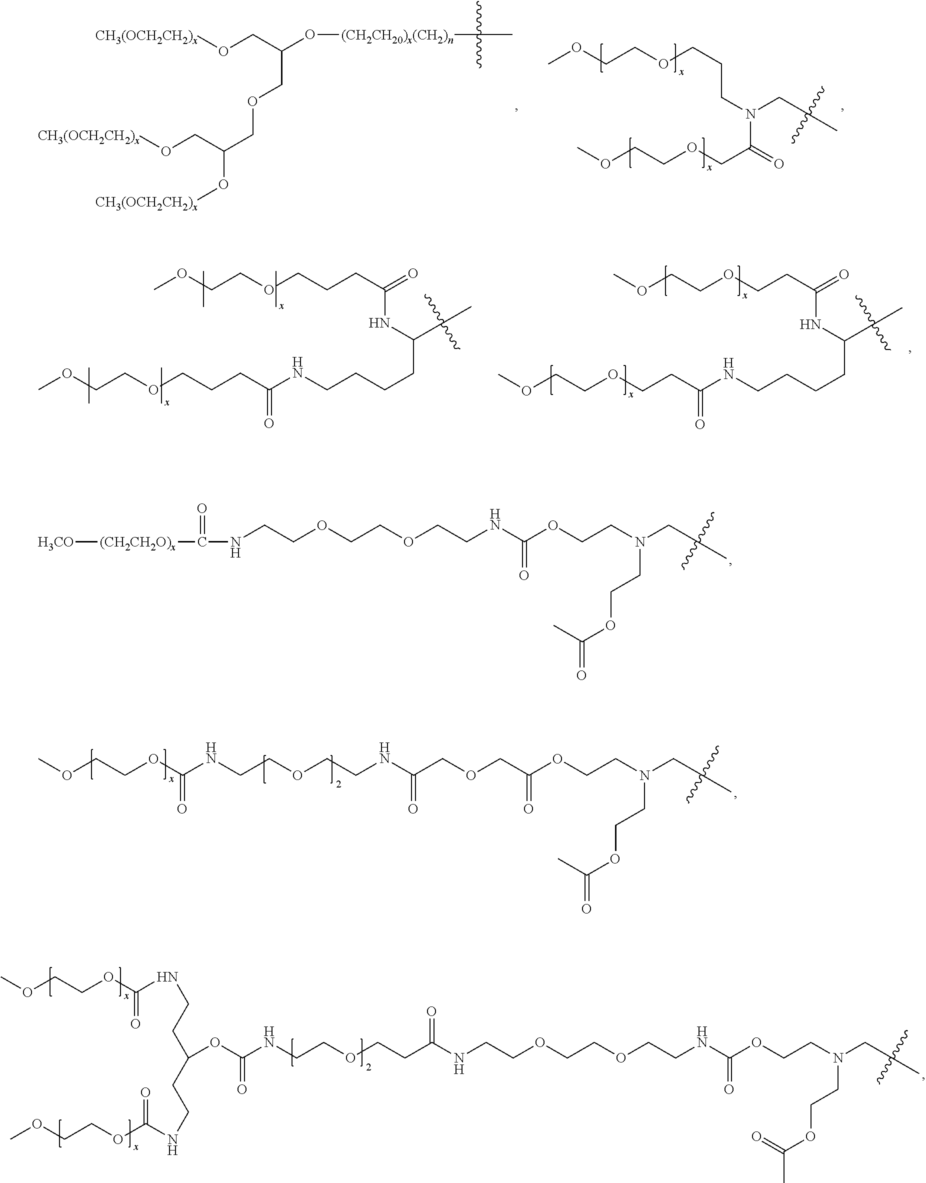 [chemical structure]
